(19) United States
(12) Patent Application Publication
(10) Pub. No.: US 2005/0107350 A1
(43) Pub. Date: May 19, 2005

(54) Method for the treatment or prevention of bone disorders with a cyclooxygenase-2 inhibitor alone and in combination with a bone disorder treatment agent and compositions therewith

(75) Inventor: Lisa Maria Olson, Creve Coeur, MO (US)

Correspondence Address: James E. Davis;Harness, Dickey & Pierce, P. L. C., 7700 Bonhomme, Suite 400, Clayton, MO, 63105, US

(73) Assignee: Pharmacia Corporation, Chesterfield, MO (US)

(21) Appl. No.: 10/917,104

(22) Filed: Aug. 12, 2004

Related U.S. Application Data
(60) Provisional application No. 60/497,416, filed on Aug. 22, 2003.

(51) Int. Cl. A61K 31/60; A61K 31/415; A61K 31/365; A61K 31/405; A61K 31/192

(52) U.S. Cl. 514/165; 514/406; 514/471; 514/602; 514/420; 514/569; 514/570

(57) ABSTRACT

The present invention describes a novel method for preventing or treating bone disorders and bone disorder-related complications in a subject involving a monotherapy with a Cox-2 inhibitor or a combination therapy with a Cox-2 inhibitor and a bone disorder treatment agent. Also described are therapeutic compositions comprising a Cox-2 inhibitor and a bone disorder treatment agent. Pharmaceutical compositions and kits for implementing the present method are also described.

### CROSS-REFERENCE TO RELATED APPLICATIONS

This application is related to and claims the priority benefit of U.S. Provisional Patent Application Ser. No. 60/497,416 filed Aug. 22, 2003, which is incorporated by reference herein in its entirety.

### BACKGROUND OF THE INVENTION

(1) Field of the Invention

The present invention relates generally to the use of an enzyme inhibitor alone and in combination with a treatment agent for the treatment or prevention of bone disorders and bone disorder-related complications, and in particular to the use of a Cox-2 inhibitor alone and in combination with a bone disorder treatment agent.

(2) Description of Related Art

Osteoporosis, the most prevalent bone disorder in America, currently affects an estimated 20 million people, with another 34 million Americans having a low bone mass, thereby placing them at a heightened risk of osteoporosis in the future. Osteoporosis accounts for 1.5 million bone fractures every year, with roughly 85% of those fractures occurring in the patient's hip, spine, or wrist.

Hip fractures can be particularly harmful, since only 15% of patients can walk across a room unaided 6 months after the fracture. Furthermore, an average of 24% of all hip fracture patients over the age of 50 die within a year of the fracture. Roughly 80% of those affected by osteoporosis are post-menopausal women, and one out of every four women will suffer from osteoporosis during their lifetime. Osteoporosis is also costly, as $17 billion were spent for hospital and nursing home care for osteoporosis-related bone fractures in 2001 alone.

In some instances, prevention of osteoporosis can be more effective than treatment and may only require that a person consume an adequate amount of calcium and vitamin D, as well as taking part in some weight-bearing exercise. However, this is often not enough for post-menopausal women because estrogen levels decrease significantly during the five years following the onset of menopause.

While adequate consumption of calcium and vitamin D can help prevent osteoporosis, smoking and excessive alcohol consumption can have the opposite effect. For women, smoking results in earlier menopause and significant bone loss. For men, heavy alcohol intake is one of the leading causes of osteoporosis and bone fracture. Prevention is made more difficult by the fact that in the majority of cases, there are no symptoms of osteoporosis and, as a result, most people are not aware of the problem until a fracture has occurred and treatment is required.

There are currently two main types of pharmaceutical therapy for the treatment of osteoporosis. The first, and most common, is the use of antiresorptive compounds to reduce the resorption of bone tissue, thereby preventing deterioration of the skeletal structure. Estrogen replacement therapy (“ERT”) is an example of an antiresorptive treatment. ERT is known to prevent further deterioration and thereby reduce the likelihood of fractures. However, the use of estrogen as a treatment is limited, as it is believed that long-term estrogen therapy may lead to an increase in the risk of uterine cancer, endometrial cancer, frequent vaginal bleeding, and the potential for thrombosis. Further, studies have suggested that ERT may increase a woman's risk of breast cancer by as much as 25%. The threat of endometrial cancer may be offset by consuming progesterone, although such consumption diminishes the favorable effect of the estrogen. Because of the serious side effects, many women choose to avoid this treatment. Further, few men agree to this type of treatment.

Bisphosphonates, particularly alendronate and risedronate, are currently the most potent antiresorptive treatments for osteoporosis. Tests have shown that these compounds consistently increase the bone mineral density (“BMD”) in osteoporosis patients. BMD is the best indicator of whether a patient is at risk of bone fracture due to osteoporosis. Although benefits have been realized through these treatments, the exact process by which this occurs has yet to be precisely defined. See Hodsman, D., et al., CMAJ, 166:1426 (2002). Studies have suggested that as many as 95% of patients treated with alendronate show an increase in BMD at the lumbar spine. On average, a patient's BMD has improved by 8% after three years of treatment. Further, in women with prevalent vertebral fractures, both alendronate and risedronate unequivocally reduce incident fractures at all sites by up to 50% when treatment continues for 3-4 years or longer.

However, there are significant problems with the treatment of osteoporosis by bisphosphonates. To ensure that alendronate is absorbed, it must be swallowed with a full glass of water immediately upon arising for the day, and no other food or drink should be taken for the next 30 minutes. Because alendronate can irritate the lining of the upper gastrointestinal tract, the patient must not lie down after taking a dose for at least the next 30 minutes, and until they have had something to eat. Because of this required routine, many patients cannot diligently continue treatment under this program. Further, certain people with difficulty swallowing or with certain disorders of the esophagus or stomach cannot use this treatment.

Calcitonin may also be used to treat osteoporosis, although it is probably the least potent of the available treatments. Calcitonin is a 32-amino-acid peptide that directly inhibits osteoclasts, thereby slowing bone loss. However, calcitonins are broken down by amino peptidases and proteases in the gastrointestinal tract and,.therefore, they cannot be administered orally. As a result, calcitonin must be injected, which is often inconvenient for patients, or absorbed as a nasal spray, which is widely accepted by patients but is not as effective as when injected. Reports suggest that intranasal calcitonin may improve BMD by 1-2% after 2 years of treatment. Calcitonin does not come without side effects, although the common effects are relatively minor and include flushing, nausea, vomiting, and diarrhea.

Osteoporosis can also be treated by raloxifene. Raloxifene is a selective estrogen receptor modulator (SERM) that has beneficial effects on bone as well as endometrial and breast tissue. Studies suggest that raloxifene can increase the BMD of the lumbar spine and hip by 2-3% over 2-3 years of treatment, and decrease vertebral fractures by 50%. Further, raloxifene significantly decreases the risk of breast cancer.

The side effects of raloxifene are relatively mild, except in rare instances in which blood clotting has been reported. The most common side effects associated with raloxifene are leg cramps and hot flashes.

A second type of pharmaceutical therapy for the treatment of osteoporosis is the use of anabolic agents to promote bone formation and increase bone mass, rather than simply to prevent deterioration. The only anabolic agent currently available is parathyroid hormone (PTH). Studies have suggested that such treatment increases the BMD in the spine by as much as 13%. See Neer, M., et al, NEJM, 344:1434-1441 (2001). However, patients must inject the medication daily and common side effects of this treatment include nausea, headaches, and leg cramps. This treatment has been shown to cause an increase in bone tumors in rats, although it has yet to be determined whether the same effect would occur in humans.

Paget's disease (osteitis deformans), a disorder that is related to osteoporosis, is also the result of abnormal bone regeneration. Paget's disease affects around 3% of Americans, while the prevalence of familial Paget's disease (where more than one family member has the disease) ranges from 10 to 40 percent in different parts of the world. Paget's disease affects men and women equally and generally affects people over 40 years of age.

In a few instances, it has been reported that Cox-2 inhibitors may be effective for the treatment of bone disorders. U.S. Pat. No. 5,663,195 discloses the treatment of osteoporosis with the Cox-2 selective inhibitor, rofecoxib and its derivatives. Other reports, however, indicate that Cox-2 inhibitors interfere with the healing of broken bones, implying that Cox-2 inhibitors would not be an appropriate treatment of bone disorders or bone disorder-related complications. See Goodman, S., et al., J. of Orthopaedic Res. 16:1164-1169 (2002). Studies have also suggested that a bone that has completely healed after fracture may be weaker if a Cox-2 inhibitor is administered during the healing period. See Simon, A., et al., J. Bone Mineral Res. 17:963-976 (2002).

Unfortunately, even with the multitude of bone disorder treatments that are now available or in clinical trials, osteoporosis and other bone disorders remain difficult to treat effectively given the side effects of, and demanding routine required by, current treatments. It would be useful, therefore, to provide efficacious methods and compositions for the prevention and treatment of bone disorders and bone disorder-related complications, while reducing the dosage and unwanted side effects that are present in current treatments. Such reduction in dosage would not only decrease the quantity and severity of the side effects of current treatments, but it would also significantly reduce the cost of treatment, including hospital and nursing home expenditures. From the foregoing, it can be seen that a need exists for improved methods and compositions for preventing and treating bone disorders and bone disorder-related complications.

### SUMMARY

Briefly, therefore, the present invention is directed to a novel method of preventing or treating bone disorders and bone disorder-related complications in a subject comprising administering to the subject a Cox-2 inhibitor.

The present invention is also directed to a novel method of preventing or treating bone disorders and bone disorder-related complications in a subject that is in need of such prevention or treatment comprising administering to the subject a Cox-2 inhibitor.

The present invention is also directed to a method of preventing or treating bone disorders and bone disorder-related complications in a subject comprising administering to the subject a Cox-2 inhibitor in combination with a bone disorder treatment agent.

The present invention is also directed to a method of preventing or treating bone disorders and bone disorder-related complications in a subject in need of such prevention or treatment comprising administering to the subject a Cox-2 inhibitor in combination with a bone disorder treatment agent.

The present invention is also directed to a novel therapeutic composition comprising a Cox-2 inhibitor and a bone disorder treatment agent.

The present invention is also directed to a pharmaceutical composition comprising a Cox-2 inhibitor, a bone disorder treatment agent, and a pharmaceutically acceptable carrier.

The present invention is also directed to a novel kit comprising one dosage form comprising a Cox-2 inhibitor and a second dosage form comprising a bone disorder treatment agent.

Among the several advantages found to be achieved by the present invention, therefore, may be noted the provision of improved treatment methods and compositions for bone disorders, the provision of such improved methods and compositions comprising Cox-2 inhibitors alone and in combination with one or more conventional bone disorder treatment agents that are useful for treating and preventing bone disorders and bone disorder-related complications.

### DETAILED DESCRIPTION OF THE PREFERRED EMBODIMENTS

In accordance with the present invention, it has been discovered that the treatment or prevention of bone disorders and bone disorder-related complications, including such disorders as osteoporosis, is provided by either a monotherapy comprising a Cox-2 inhibitor or a combination therapy comprising a Cox-2 inhibitor and a bone disorder treatment agent.

For purposes of the present invention, the novel monotherapy or combination therapy comprising a Cox-2 inhibitor alone or in combination with a bone disorder treatment agent is useful for the purpose of preventing and treating bone disorders and bone disorder-related complications in a subject and, in preferred embodiments, the method is useful for the purpose of preventing and treating bone disorders and bone disorder-related complications in a subject that is in need of such prevention or treatment.

The methods and compositions of the present invention would be useful, for example, to reduce such bone disorder symptoms as skeletal distortion and low bone mineral density in a subject suffering from such symptoms. The monotherapy or combination therapy of the present invention would also be useful to prevent the occurrence of such symptoms.

The monotherapy and combination therapy of the present invention are also useful for the treatment of bone disorder-related complications, which may arise indirectly from having a bone disorder, by treating the underlying bone disorder itself. For example, if a subject is suffering from a bone disorder-related complication, such as a bone fracture, the treatment of the underlying bone disorder, such as osteopenia or osteoporosis, by the methods and compositions of the present invention will likewise improve the symptoms of the associated complication.

The methods and compositions of the present invention are also useful to reduce the number of hospitalizations of subjects suffering from bone related disorders, or to prevent or retard, in subjects, the development of complications associated with bone disorders, such as, for example, bone fractures and/or bone breaks, which may eventually arise from having osteoporosis and other bone disorders.

The administration of a Cox-2 inhibitor for the prevention or treatment of bone disorders and bone disorder-related complications is an unexpectedly effective treatment and preventative therapy. Such administration is effective for improving the symptoms of bone disorders and bone disorder-related complications while avoiding or reducing certain disadvantages of current treatments.

Combination therapies comprising Cox-2 inhibitors and bone disorder treatment agents are useful not only for improving bone disorder symptoms and shortening recovery times, but also for reducing the dosages of bone disorder treatment agents that are normally required. Reduced dosages of bone disorder treatment agents are beneficial where normal dosages exhibit harmful side effects or require burdensome treatment regimens, for example, as with some conventional osteoporosis treatment agents such as estrogen replacement therapy and bisphosphonates. The administration of low dosages of conventional bone disorder treatment agents can, in one embodiment, provide a reduction in side effects corresponding to such agents.

As used herein, the terms “lowered dosages”, “low dose”, or “low dose amount”, in characterizing a therapeutically effective amount of a Cox-2 inhibitor alone or in combination with a bone disorder treatment agent defines a quantity of such agent, or a range of quantity of such agent, that is capable of preventing or treating the symptoms of a bone disorder or a bone disorder-related complication while optionally reducing or avoiding one or more side effects of a monotherapy with a conventional bone disorder treatment agent.

The combination therapy of a Cox-2 inhibitor and a bone disorder treatment agent may also be useful for decreasing the required number of separate dosages, thus, potentially improving patient compliance. For example, estrogen replacement therapy has extensive side effects and bisphosphonates not only require an extremely burdensome treatment regimen, but can also irritate the lining of the upper gastrointestinal tract. Therefore, in one embodiment, the therapies of the present invention are useful for reducing the dosing frequency of conventional bone disorder treatment agents such as estrogen replacement drugs and bisphosphonates. Administering the therapies of the present invention to a subject undergoing multiple dosing with a conventional bone disorder treatment agent may also reduce the required number of separate doses of the bone disorder treatment agent normally prescribed.

The administration of a Cox-2 inhibitor in combination with a bone disorder treatment agent is an effective treatment for bone disorders and bone disorder-related complications, and in preferred embodiments, is superior to the use of either agent alone. Moreover, in preferred embodiments, the combination therapies of the present invention demonstrate a synergistic efficacy for treating and preventing bone disorders and bone disorder-related complications that is greater than what would be expected from simply combining any of the individual monotherapies. As used herein, the term “synergistic” refers to the combination of a Cox-2 inhibitor and a bone disorder treatment agent as a combined therapy having an efficacy for the prevention and treatment of bone disorders that is greater than what would be expected merely from the sum of their individual effects. The synergistic effects of the embodiments of the present invention's combination therapies encompass additional unexpected advantages for the treatment and prevention of bone disorders. Such additional advantages include, but are not limited to, lowering the required dose of bone disorder treatment agents, reducing the side-effects of bone disorder treatment agents, and rendering those agents more tolerable to subjects in need of bone disorder therapy.

As used herein, the term “monotherapy” is intended to embrace administration of a Cox-2 inhibitor to a subject suffering from a bone disorder or bone disorder-related complication as a single therapeutic treatment without an additional therapeutic treatment comprising a bone disorder treatment agent. However, the Cox-2 inhibitor may still be administered in multiple dosage forms. Thus, the Cox-2 inhibitor may be administered in one therapeutic dosage form, such as in a single capsule, tablet, or injection, or in two separate therapeutic dosage forms, such as in separate capsules, tablets, or injections.

As used herein, the terms “combination therapy”, “co-administration”, “co-administering”, “administration with”, “administering”, “combination”, or “co-therapy”, when referring to the use of a Cox-2 inhibitor in combination with a bone disorder treatment agent, are intended to embrace administration of each agent in a sequential manner in a regimen that will provide beneficial effects of the drug combination, and is intended as well to embrace co-administration of these agents in a substantially simultaneous manner.

Substantially simultaneous administration can be accomplished, for example, by administering to the subject the Cox-2 inhibitor in combination with a bone disorder treatment agent, together in one therapeutic dosage form, such as in a single capsule, tablet, or injection, or in multiple separate therapeutic dosage forms, such as in separate capsules, tablets, or injections.

Sequential or substantially simultaneous administration of each therapeutic agent can be effected by any appropriate route including, but not limited to, oral routes, intravenous routes, intramuscular routes, subcutaneous routes, intraarticular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

The phrase “combination therapy” also can embrace the administration of the therapeutic agents as described above in further combination with other biologically active ingredients and non-drug therapies.

Sequential administration of such treatments encompasses both relatively short and relatively long periods between the administration of each of the compounds of the present method. However, for purposes of the present invention, the bone disorder treatment agent is administered while the Cox-2 inhibitor is still having an efficacious effect on the subject.

Preferably, the bone disorder treatment agent is to be given to the subject within the therapeutic response time of the administered Cox-2 inhibitor. As used herein, the terms “therapeutic response time” mean the duration of time after administration that a compound has a therapeutic effect within a subject's body.

As used herein, the terms “therapeutically effective” are intended to qualify the amount of an agent for use in a therapy that will achieve the goal of preventing or treating by improvement in the severity of the bone disorder symptoms or bone disorder-related complication symptoms in a subject, while avoiding adverse side effects typically associated with alternative therapies.

In one embodiment, the present invention encompasses a method for preventing bone disorders or bone disorder-related complications in a subject, and in preferred embodiments, preventing bone disorders or bone disorder-related complications in subject that is predisposed to bone disorders or bone disorder-related complications, the method comprising administering to the subject a Cox-2 inhibitor alone or in combination with a bone disorder treatment agent.

As used herein, the terms “to prevent”, “preventing”, or “prevention” refer to any reduction, no matter how slight, of a subject's predisposition or risk for developing a bone disorder or a bone disorder-related complication. For purposes of prevention, the subject is any subject, and preferably is a subject that is at risk for, or is predisposed to, developing a bone disorder or a bone disorder-related complication. As used herein, the terms “predisposition”, “predisposed to” or “at risk for,” all of which may be used interchangeably herein, includes any subject with an increased chance for developing a bone disorder or a bone disorder-related complication. The subject may be at risk due to genetic predisposition, diet, trauma, sex, age, exposure to bone disorder causing agents, and the like. The subject may also be at risk for re-developing a bone disorder or a bone disorder-related complication after suffering from a bone disorder or a bone disorder-related complication. The subject may also be at risk due to physiological factors such as anatomical and biochemical abnormalities.

In another embodiment, the present invention encompasses a method for treating bone disorders or bone disorder-related complications in a subject, and in preferred embodiments, treating bone disorders or bone disorder-related complications in subject that is suffering from a bone disorder or a bone disorder-related complication, the method comprising administering to the subject a Cox-2 inhibitor alone or in combination with a bone disorder treatment agent.

As used herein, the terms “treating”, “treatment”, “treated”, or “to treat,” mean to alleviate symptoms, eliminate the causation either on a temporary or permanent basis, or to alter or slow the appearance of symptoms or symptom worsening.

The amount of the Cox-2 inhibitor that is used in the method is selected so that it constitutes a bone disorder treatment or prevention effective amount. In those embodiments where a bone disorder treatment agent is also present, the amount of bone disorder treatment agent is selected so that when they are used in combination with the Cox-2 inhibitor, a dosage of the combination provides a bone disorder treatment or prevention effective amount.

One component of the present invention is a Cox-2 inhibitor.

Inhibitors of the Cox pathway in the metabolism of arachidonic acid may inhibit enzyme activity through a variety of mechanisms. By way of example, the Cox-2 inhibitors used in the methods described herein may block the enzyme activity directly by binding at the substrate site ,of the enzyme. In preferred embodiments, the use of a Cox-2 selective inhibitor is highly advantageous in that it minimizes the gastric side effects that can occur with non-selective non-steroidal anti-inflammatory drugs (NSAIDs), especially where prolonged treatment is expected.

The terms “cyclooxygenase-2 inhibitor”, or “Cox-2 inhibitor”, which can be used interchangeably herein, embrace compounds, which inhibit the Cox-2 enzyme regardless of the degree of inhibition of the Cox-1 enzyme, and include pharmaceutically acceptable salts of those compounds. Thus, for purposes of the present invention, a compound is considered a Cox-2 inhibitor irrespective of whether the compound inhibits the Cox-2 enzyme to an equal, greater, or lesser degree than the Cox-1 enzyme.

In one embodiment of the present invention, it is preferred that the Cox-2 inhibitor compound is a non-steroidal anti-inflammatory drug (NSAID). Therefore, preferred materials that can serve as the Cox-2 inhibitor of the present invention include non-steroidal anti-inflammatory drug compounds, a pharmaceutically acceptable salt thereof, mixed isomer, or a pure (−) or (+) optical isomeric form thereof.

Examples of NSAID compounds that are useful in the present invention include acemetacin, acetyl salicylic acid, alclofenac, alminoprofen, azapropazone, benorylate, benoxaprofen, bucloxic acid, carprofen, choline magnesium trisalicylate, clidanac, clopinac, dapsone, diclofenac, diflunisal, droxicam, etodolac, fenoprofen, fenbufen, fenclofenec, fentiazac, floctafenine, flufenisal, flurbiprofen, (r)-flurbiprofen, (s)-flurbiprofen, furofenac, feprazone, flufenamic acid, fluprofen, ibufenac, ibuprofen, indometacin, indomethacin, indoprofen, isoxepac, isoxicam, ketoprofen, ketorolac, miroprofen, piroxicam, meloxicam, mefenamic, mefenamic acid, meclofenamic acid, meclofen, nabumetone, naproxen, niflumic acid, nitroflurbiprofen, oxaprozin, oxipinac, oxyphenbutazone, phenylbutazone, podophyllotoxin derivatives, proglumetacin, piprofen, pirprofen, prapoprofen, salicylic acid, salicylate, sudoxicam, suprofen, sulindac, tenoxicam, tiaprofenic acid, tiopinac, tioxaprofen, tolfenamic acid, tolmetin, zidometacin, zomepirac, and 2-fluoro-a-methyl[1,1′-biphenyl]-4-acetic acid, a 4-(nitrooxy)butyl ester, and mixtures thereof.

Further preferred NSAID compounds include ibuprofen, naproxen, sulindac, ketoporfen, fenoprofen, tiaprofenic acid, suprofen, etodolac, carprofen, ketrolac, piprofen, indoprofen, salicylic acid, flurbiprofen, and mixtures thereof.

In a preferred embodiment, the Cox-2 inhibitor is a Cox-2 selective inhibitor. The term “Cox-2 selective inhibitor” embraces compounds, which selectively inhibit the Cox-2 enzyme over the Cox-1 enzyme, and also include pharmaceutically acceptable salts and prodrugs of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of Cox activity. Preferred Cox-2 selective inhibitors of the present invention have a Cox-2 IC₅₀ of less than about 1 μM, more preferred of less than about 0.5 μM, and even more preferred of less than about 0.2 μM.

Preferred Cox-2 selective inhibitors have a Cox-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of Cox-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic Cox-2 selective inhibitor valdecoxib. An example of a preferred Cox-2 selective inhibitor prodrug is sodium parecoxib. A class of prodrugs of Cox-2 inhibitors is described in U.S. Pat. No. 5,932,598.

In one embodiment of the present invention, the Cox-2 selective inhibitor is of the chromene/chroman (“chromene”) structural class, which encompasses substituted benzopyrans or substituted benzopyran analogs, as well as substituted benzothiopyrans, dihydroquinolines, or dihydronaphthalenes having the structure of general Formula I, shown below, and including, by way of non-limiting example, the chromene compounds described below, and the diastereomers, enantiomers, racemates, tautomers, salts, esters, amides and prodrugs thereof.

Chromenes that can serve as a Cox-2 selective inhibitor of the present invention include any one or more of the compounds that are described in U.S. Pat. Nos. 6,271,253; 6,492,390; 6,034,256 and 6,077,850. One such class of compounds is defined by the general formula shown below in formula l: 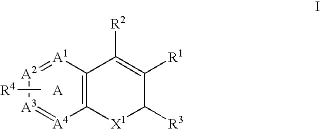
wherein X¹ is selected from O, S, CR^{c}R^{b} and NR^{a};
wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b}R^{c} forms a cycloalkyl ring;
wherein R¹ is selected from carboxyl, alkyl, aralkyl, aminocarbonyl, C₁-C₆-alkylsuIfonylaminocarbonyl and alkoxycarbonyl;
wherein R² is selected from hydrido, phenyl, thienyl, C₂-C₆-alkynyl, C₁-C₆-alkyl and C₂-C₆-alkenyl;
wherein R³ is selected from C₁-C₃-perfluoroalkyl, chloro, C₁-C₆-alkylthio, C₁-C₆-alkoxy, nitro, phenyl, cyano, cyano-C₁-C₃-alkyl, haloalkyl, alkyl, aralkyl, cycloalkyl, and aryl, wherein haloalkyl, alkyl, aralkyl, cycloalkyl, and aryl each is independently optionally substituted with one or more radicals selected from alkylthio, nitro and alkylsulfonyl;
wherein R⁴ is one or more radicals independently selected from hydrido, halo, C₁-C₆-alkyl, C₂-C₆-alkenyl, C₂-C₆-alkynyl, halo-C₂-C₆ -alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, O(CF₂)₂ O-, aryloxy, arylthio, arylsulfinyl, heteroaryloxy, aralkyloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₁-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon; or
wherein R⁴ together with ring A forms a radical selected from naphthyl, quinolyl, isoquinolyl, quinolizinyl, quinoxalinyl and dibenzofuryl; or an isomer or pharmaceutically acceptable salt thereof.

The meaning of any substituent at any one occurrence in any general chemical formula herein, is independent of its meaning, or any other substituent's meaning, at any other occurrence, unless specified otherwise.

The term “alkyl” is used, either alone or within other terms such as “haloalkyl” and “alkylsulfonyl”; it embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. The number of carbon atoms can also be expressed as “C₁-C₅”, for example. Examples of such radicals include methyl, ethyl, n-propyl, isopropyl, n-butyl, isobutyl, sec-butyl, tert-butyl, pentyl, isoamyl, hexyl, octyl and the, like. The term “alkenyl” refers to an unsaturated, acyclic hydrocarbon radical, linear or branched, in so much as it contains at least one double bond. Unless otherwise noted, such radicals preferably contain from 2 to about 6 carbon atoms, preferably from 2 to about 4 carbon atoms, more preferably from 2 to about 3 carbon atoms. The alkenyl radicals may be optionally substituted with groups as defined below. Examples of suitable alkenyl radicals include propenyl, 2-chloropropylenyl, buten-1yl, isobutenyl, penten-1yl, 2-methylbuten-1-yl, 3-methylbuten-1-yl, hexen-1-yl, 3-hydroxyhexen-1-yl, hepten-1-yl, octen-1-yl, and the like. The term “alkynyl” refers to an unsaturated, acyclic hydrocarbon radical, linear or branched, in so much as it contains one or more triple bonds, such radicals preferably containing 2 to about 6 carbon atoms, more preferably from 2 to about 3 carbon atoms. The alkynyl radicals may be optionally substituted with groups as described below. Examples of suitable alkynyl radicals include ethynyl, proynyl, hydroxypropynyl, butyn-1-yl, butyn-2-yl, pentyn-1-yl, pentyn-2-yl, 4-methoxypentyn-2-yl, 3-methylbutyn-1-yl, hexyl-1-yl, hexyn-2-yl, hexyn-3-yl, 3,3-dimethylbutyn-1-yl radicals, and the like.

The term “oxo” means a single double-bonded oxygen. The terms “hydrido”, “—H”, or “hydrogen”, denote a single hydrogen atom (H). This hydrido radical may be attached, for example, to an oxygen atom to form a hydroxyl radical, or two hydrido radicals may be attached to a carbon atom to form a methylene (—CH₂ —) radical.

The term “halo” means halogens such as fluorine, chlorine, and bromine or iodine atoms. The term “haloalkyl” embraces radicals wherein any one or more of the alkyl carbon atoms is substituted with halo as defined above. Specifically embraced are monohaloalkyl, dihaloalkyl, and polyhaloalkyl radicals. A monohaloalkyl radical, for one example, may have a bromo, chloro, or a fluoro atom within the radical. Dihalo radicals may have two or more of the same halo atoms or a combination of different halo radicals and polyhaloalkyl radicals may have more than two of the same halo atoms or a combination of different halo radicals. Likewise, the term “halo”, when it is appended to alkenyl, alkynyl, alkoxy, aryl, cycloalkyl, heteroalkyl, heteroaryl, and the like, includes radicals having mono-, di-, or tri-, halo substitution on one or more of the atoms of the radical.

The term “hydroxyalkyl” embraces linear or branched alkyl radicals having one to about ten carbon atoms any one of which may be substituted with one or more hydroxyl radicals.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and diaikoxyalkyl radicals. The “alkoxy” or “alkoxyalkyl” radicals may be further substituted with one or more halo atoms, such as fluoro, chloro, or bromo, to provide “haloalkoxy” or “haloalkoxyalkyl” radicals. Examples of “alkoxy” radicals include methoxy, butoxy, and trifluoromethoxy. Terms such as “alkoxy(halo)alkyl”, indicate a molecule having a terminal alkoxy that is bound to an alkyl, which is bonded to the parent molecule, while the alkyl also has a substituent halo group in a non-terminal location. In other words, both the alkoxy and the halo group are substituents of the alkyl chain.

The term “aryl”, alone or in combination, means a carbocyclic aromatic system containing one, two, or three rings wherein such rings may be attached together in a pendent manner or may be fused. The term “aryl” embraces aromatic radicals such as phenyl, naphthyl, tetrahydronapthyl, indane, and biphenyl. The term “heterocyclyl” means a saturated or unsaturated mono- or multi-ring carbocycle wherein one or more carbon atoms is replaced by N, S, P, or O. This includes, for example, structures such as: 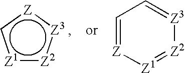
where Z, Z¹, Z², or Z³ is C, S, P, O, or N, with the proviso that one of Z, Z¹, Z², or Z³ is other than carbon, but is not O or S when attached to another Z atom by a double bond or when attached to another O or S atom. Furthermore, the optional substituents are understood to be attached to Z, Z¹, Z², or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others. The term “heteroaryl” embraces unsaturated heterocyclic radicals. Examples of unsaturated heterocyclic radicals, also termed “heteroaryl” radicals include thienyl, pyrryl, furyl, pyridyl, pyrimidyl, pyrazinyl, pyrazolyl, oxazolyl, isoxazolyl, imidazolyl, thiazolyl, pyranyl, and tetrazolyl. The term also embraces radicals where heterocyclic radicals are fused with aryl radicals. Examples of such fused bicyclic radicals include benzofuran, benzothiophene, and the like. The terms aryl or heteroaryl, as appropriate, include the following structures: 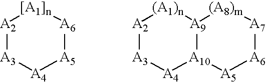
   where:

when n=1, m=1 and A₁-A₈ are each CR^{x} or N, A₉ and A₁₀ are carbon;
when n=0, or 1, and m=0, or 1, one of A₂-A₄ and/or A₅-A₇ is optionally S, O, or NR^{x}, and other ring members are CR^{x} or N, with the proviso that oxygen cannot be adjacent to sulfur in a ring. A₉ and A₁₀ are carbon;
when n is greater than or equal to 0, and m is greater than or equal to 0, 1 or more sets of 2 or more adjacent atoms A₁-A₁₀ are sp3 O, S, NR^{x}, CR^{x}R^{y}, or C═(O or S), with the proviso that oxygen and sulfur cannot be adjacent. The remaining A₁-A₈ are CR^{x} or N, and A₉ and A₁₀ are carbon;
when n is greater than or equal to 0, and m is greater than or equal to 0, atoms separated by 2 atoms (i.e., A₁ and A₄) are sp3 O, S, NR^{x}, CR^{x}R^{y}, and remaining A₁-A₈ are independently CR^{x} or N, and A₉ and A₁₀ are carbon.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂—NH₂), which may also be termed an “aminosulfonyl”. The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical.

The terms “carboxy” or “carboxyl”, whether used alone or with other terms, such as “carboxyalkyl”, denotes —CO₂—H. The term “carboxyalkyl” embraces radicals having a carboxyradical as defined above, attached to an alkyl radical. The term “carbonyl”, whether used alone or with other terms, such as “alkylcarbonyl”, denotes —(C=O)—. The term “alkylcarbonyl” embraces radicals having a carbonyl radical substituted with an alkyl radical. An example of an “alkylcarbonyl” radical is CH₃—(CO)—. The term “alkylcarbonylalkyl” denotes an alkyl radical substituted with an “alkylcarbonyl” radical. The term “alkoxycarbonyl” means a radical containing an alkoxy radical, as defined above, attached via an oxygen atom to a carbonyl (C═O) radical. Examples of such “alkoxycarbonyl” radicals include (CH₃)₃—C—O—C═O)— and —(O═)C—OCH₃. The term “alkoxycarbonylalkyl” embraces radicals having “alkoxycarbonyl”, as defined above substituted to an alkyl radical. Examples of such “alkoxycarbonylalkyl” radicals include (CH₃)₃C—OC(═O)—(CH₂)₂— and —(CH₂)₂(—O)COCH₃. The terms “amido”, or “carbamyl”, when used alone or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkylradical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkylradicals substituted with an N-alkyl-N-hydroxyamido radical. The term “amidoalkyl” embraces alkyl radicals substituted with amido radicals. The term “aminoalkyl” embraces alkyl radicals substituted with amino radicals. The term “alkylaminoalkyl” embraces aminoalkyl radicals having the nitrogen atom substituted with an alkyl radical. The term “amidino” denotes an —C(—NH)—NH₂ radical. The term “cyanoamidin” denotes an —C(—N—CN)—NH₂ radical. The term “heterocycloalkyl” embraces heterocyclic-substituted alkyl radicals such as pyridylmethyl and thienylmethyl.

The terms “aralkyl”, or “arylalkyl” embrace aryl-substituted alkyl radicals such as benzyl, diphenylmethyl, triphenylmethyl, phenethyl, and diphenethyl. The terms benzyl and phenylmethyl are interchangeable. The term “cycloalkyl” embraces radicals having three to ten carbon atoms, such as cyclopropyl cyclobutyl, cyclopentyl, cyclohexyl, and cycloheptyl. The term “cycloalkenyl” embraces unsaturated radicals having three to ten carbon atoms, such as cylopropenyl, cyclobutenyl, cyclopentenyl, cyclohexenyl, and cycloheptenyl. The term “alkylthio” embraces radicals containing a linear or branched alkyl radical, of one to ten carbon atoms, attached to a divalent sulfur atom. An example of “alkylthio” is methylthio, (CH₃—S—). The term “alkylsulfinyl” embraces radicals containing a linear or branched alkyl radical, of one to ten carbon atoms, attached to a divalent —S(—O)— atom. The terms “N-alkylamino” and “N, N-dialkylamino” denote amino groups which have been substituted with one alkyl radical and with two alkyl radicals, respectively.

The term “acyl”, whether used alone, or within a term such as “acylamino”, denotes a radical provided by the residue after removal of hydroxyl from an organic acid. The term “acylamino” embraces an amino radical substituted with an acyl group. An examples of an “acylamino” radical is acetylamino (CH₃—C(═O)—NH—).

In the naming of substituent groups for general chemical structures, the naming of the chemical components of the group is typically from the terminal group-toward the parent compound unless otherwise noted, as discussed below. In other words, the outermost chemical structure is named first, followed by the next structure in line, followed by the next, etc. until the structure that is connected to the parent structure is named. For example, a substituent group having a structure such as: 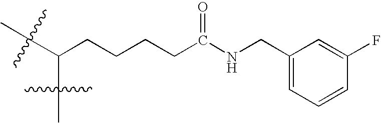
may be referred to generally as a “haloarylalkylaminocarboxylalkyl”. An example of one such group would be fluorophenylmethylcarbamylpentyl. The bonds having wavy lines through them represent the parent structure to which the alkyl is attached.

Substituent groups may also be named by reference to one or more “R” groups. The structure shown above would be included in a description, such as, “—C₁-C6-alkyl-COR^{u}, where R^{u} is defined to include—NH—C₁-C4-alkylaryl-R^{y}, and where R^{y} is defined to include halo. In this scheme, atoms having an “R” group are shown with the “R” group being the terminal group (i.e., furthest from the parent). In a term such as “C(R^{x})₂”, it should be understood that the two R^{x} groups can be the same, or they can be different if R^{x} is defined as having more than one possible identity.

Examples of chromene Cox-2 inhibitors that are suitable for use with the methods and compositions of the present invention include any one or more of:
6-nitro-2-trifluoromethyl-2H-1-benzopyran-3-carboxylic acid; 6-chloro-8-methyl-2-trifluoromethyl-2H-1-benzopyran-3-carboxylic acid; (S)-6-chloro-7-(1,1-dimethylethyl)-2-trifluoromethyl-2H-1-benzopyran-3-carboxylic acid; 2-trifluoromethyl-2H-naphthol[2,3-b]pyran-3-carboxylic acid; 6-chloro-7-(4-nitrophenoxy)-2-(trifluoromethyl)-2H-1-benzopyran-3-carboxylic acid; (S)-6,8-dichloro-2-(trifluoromethyl)-2H-1-benzopyran-3-carboxylic acid; 6-chloro-2-(trifluoromethyl)-4-phenyl-2H-1-benzopyran-3-carboxylic acid; 6-(4-hydroxybenzoyl)-2-(trifluoromethyl)-2H-1-benzopyran-3-carboxylic acid; 2-(trifluoromethyl)-6-[(trifluoromethyl)thiol]-2H-1-benzothiopyran-3-carboxylic acid; 6,8-dichloro-2-trifluoromethyl-2H-1-benzothiopyran-3 carboxylic acid; 6-(1,1-dimethylethyl)-2-(trifluoromethyl)-2H-1-benzothiopyran-3-carboxylic acid; 6,7-difluoro-1,2-dihydro-2-(trifluoromethyl)-3-quinolinecarboxylic acid; 6-chloro-1,2-dihydro-1-methyl-2-(trifluoromethyl)-3-quinolinecarboxylic acid; 6-chloro-2-(trifluoromethyl)-1,2-dihydro[1,8]naphthyridine-3-carboxylic acid; (S)-6-chloro-1,2-dihydro-2-(trifluoromethyl)-3-quinolinecarboxylic acid; (2S)-6,8-dimethyl-2-(trifluoromethyl)-2H-chromene-3-carboxylic acid; (2S)-8-ethyl-6-(trifluoromethoxy)-2-(trifluoromethyl)-2H-chromene-3-carboxylic acid; (2S)-6-chloro-5,7-dimethyl-2-(trifluoromethyl)-2H-chromene-3-carboxylic acid; 6-chloro-2-trifluoromethyl-2H-1-benzopyran-3-carboxylic acid; 6-chloro-7-methyl-2-trifluoromethyl-2H-1-benzopyran-3-carboxylic acid; 8-(1-methylethyl)-2-trifluoromethyl-2H-1-benzopyran-3-carboxylic acid; 6-chloro-7-(1,1-dimethylethyl)-2-trifluoromethyl-2H-1-benzopyran-3-carboxylic acid; 6-chloro-8-(1-methylethyl)-2-trifluoromethyl-2H-1-benzopyran-3-carboxylic acid; 2-trifluoromethyl-3H-naphthopyran-3-carboxylic acid; 7-(1,1-dimethylethyl)-2-trifluoromethyl-2H-1-benzopyran-3-carboxylic acid; 6-bromo-2-trifluoromethyl-2H-1-benzopyran-3-carboxylic acid; 8-chloro-2-trifluoromethyl-2H-1-benzopyran-3-carboxylic acid; 6-trifluoromethoxy-2-trifluoromethyl-2H-1-benzopyran-3-carboxylic acid; 5,7-dichloro-2-trifluoromethyl-2H-1-benzopyran-3-carboxylic acid; 8-phenyl-2-trifluoromethyl-2H-1-benzopyran-3-carboxylic acid; 7,8-dimethyl-2-trifluoromethyl-2H-1-benzopyran-3-carboxylic acid; 6,8-bis(dimethylethyl)-2-trifluoromethyl-2H-1-benzopyran-3-carboxylic acid; 7-(1-methylethyl)-2-trifluoromethyl-2H-1-benzopyran-3-carboxylic acid; 7-phenyl-2-trifluoromethyl-2H-1-benzopyran-3-carboxylic acid; 6-chloro-7-ethyl-2-trifluoromethyl-2H-1-benzopyran-3-carboxylic acid; 6-chloro-8-ethyl-2-trifluoromethyl-2H-1-benzopyran-3-carboxylic acid; 6-chloro-7-phenyl-2-trifluoromethyl-2H-1-benzopyran-3-carboxylic acid; 6,7-dichloro-2-trifluoromethyl-2H-1-benzopyran-3-carboxylic acid; 6,8-dichloro-2-trifluoromethyl-2H-1-benzopyran-3-carboxylic acid; 2-trifluoromethyl-3H-naptho[2,1-b]pyran-3-carboxylic acid; 6-chloro-8-methyl-2-trifluoromethyl-2H-1-benzopyran-3-carboxylic acid; 8-chloro-6-methyl-2-trifluoromethyl-2H-1-benzopyran-3-carboxylic acid; 8-chloro-6-methoxy-2-trifluoromethyl-2H-1-benzopyran-3-carboxylic acid; 6-bromo-8-chloro-2-trifluoromethyl-2H-1-benzopyran-3-carboxylic acid; 8-bromo-6-fluoro-2-trifluoromethyl-2H-1-benzopyran-3-carboxylic acid; 8-bromo-6-methyl-2-trifluoromethyl-2H-1-benzopyran-3-carboxylic acid; 8-bromo-5-fluoro-2-trifluoromethyl-2H-1-benzopyran-3-carboxylic acid; 6-chloro-8-fluoro-2-trifluoromethyl-2H-1-benzopyran-3-carboxylic acid; 6-bromo-8-methoxy-2-trifluoromethyl-2H-1-benzopyran-3-carboxylic acid; 6-[[(phenylmethyl)amino]sulfonyl]-2-trifluoromethyl-2H-1-benzopyran-3-carboxylic acid; 6-[(dimethylamino)sulfonyl]-2-trifluoromethyl-2H-1-benzopyran-3-carboxylic acid; 6-[(methylamino)sulfonyl]-2-trifluoromethyl-2H-1-benzopyran-3-carboxylic acid; 6-[(4-morpholino)sulfonyl]-2-trifluoromethyl-2H-1-benzopyran-3-carboxylic acid; 6-[(1,1-dimethylethyl)aminosulfonyl]-2-trifluoromethyl-2H-1-benzopyran-3-carboxylic acid; 6-[(2-methylpropyl)aminosulfonyl]-2-trifluoromethyl-2H-1-benzopyran-3-carboxylic acid; 6-methylsulfonyl-2-trifluoromethyl-2H-1-benzopyran-3-carboxylic acid; 8-chloro-6-[[(phenylmethyl)amino]sulfonyl]-2-trifluoromethyl-2H-1-benzopyran-3-carboxylic acid; 6-phenylacetyl-2-trifluoromethyl-2H-1-benzopyran-3-carboxylic acid; 6,8-dibromo-2-trifluoromethyl-2H-1-benzopyran-3-carboxylic acid; 8-chloro-5,6-dimethyl-2-trifluoromethyl-2H-1-benzopyran-3-carboxylic acid; 6,8-dichloro-(S)-2-trifluoromethyl-2H-1-benzopyran-3-carboxylic acid; 6-benzylsulfonyl-2-trifluoromethyl-2H-1-benzopyran-3-carboxylic acid; 6-[[N-(2-furylmethyl)amino]sulfonyl]-2-trifluoromethyl-2H-1-benzopyran-3-carboxylic acid; 6-[[N-(2-phenylethyl)amino]sulfonyl]-2-trifluoromethyl-2H-1-benzopyran-3-carboxylic acid; 6-iodo-2-trifluoromethyl-2H-1-benzopyran-3-carboxylic acid; 7-(1,1-dimethylethyl)-2-pentafluoroethyl-2H-1-benzopyran-3-carboxylic acid; 6-chloro-2-trifluoromethyl-2H-1-benzothiopyran-3-carboxylic acid; 6-chloro-2-trifluoromethyl-2H-1-benzopyran-3-carboxylic acid; (S)-6-chloro-2-trifluoromethyl-2H-1-benzopyran-3-carboxylic acid; 6-chloro-7-(1,1-dimethylethyl)-2-trifluoromethyl-2H-1-benzopyran-3-carboxylic acid; (S)-6-chloro-7-(1,1-dimethylethyl)-2-(trifluoromethyl)-2H-1-benzopyran-3-carboxylic acid; 6-trifluoromethoxy-2-trifluoromethyl-2H-1-benzopyran-3-carboxylic acid; (S)-6-trifluoromethoxy-2-trifluoromethyl-2H-1-benzopyran-3-carboxylic acid; 6-formyl-2-(trifluoromethyl)-2H -1-benzopyran-3-carboxylic acid; 6-(difluoromethyl)-2-(trifluoromethyl)-2H-1-benzopyran-3-carboxylic acid; 6,8-dichloro-7-methyl-2-(trifluoromethyl)-2H-1-benzopyran-3-carboxylic acid; 6,8-dichloro-2-trifluoromethyl-2H-1-benzopyran-3-carboxylic acid; (S)-6,8-dichloro-2-(trifluoromethyl)-2H-1-benzopyran-3-carboxylic acid; 6-chloro-1,2-dihydro-2-(trifluoromethyl)-3-quinolinecarboxylic acid; (S)-6-chloro-1,2-dihydro-2-(trifluoromethyl)-3-quinolinecarboxylic acid; 6,8-dichloro-1,2-dihydro-2-(trifluoromethyl)-3-quinolinecarboxylic acid; 7-(1,1-dimethylethyl)-2-trifluoromethyl-2H-1-benzopyran-3-carboxylic acid; 6,7-dichloro-2-trifluoromethyl-2H-1-benzopyran-3-carboxylic acid; 5,6-dichloro-2-(trifluoromethyl)-2H-1-benzopyran-3-carboxylic acid; 2,6-bis(trifluoromethyl)-2H-1-benzopyran-3-carboxylic acid; 5,6,7-trichloro-2-(trifluoromethyl)-2H-1-benzopyran-3-carboxylic acid; 6,7,8-trichloro-2-(trifluoromethyl)-2H-1-benzopyran-3-carboxylic acid; 6-iodo-1,2-dihydro-2-(trifluoromethyl)-3-quinolinecarboxylic acid; 6-bromo-1,2-dihydro-2-(trifluoromethyl)-3-quinolinecarboxylic acid; 6-chloro-7-methyl-2-(trifluoromethyl)-2H-1-benzothiopyran-3-carboxylic acid; 6,8-dichloro-2-trifluoromethyl-2H-1-benzothiopyran-3-carboxylic acid; prodrugs thereof, salts thereof; isomers thereof; and/or mixtures thereof.

Further examples of preferred chromene Cox-2 inhibitors include (S)-6-chloro-7-(1,1-dimethylethyl)-2-(trifluoromethyl)-2H-1-benzopyran-3-carboxylic acid, (2S)-6,8-dimethyl-2-(trifluoromethyl)-2H-chromene-3-carboxylic acid, (2S)-6-chloro-8-methyl-2-(trifluoromethyl)-2H-chromene-3-carboxylic acid, (2S)-8-ethyl-6-(trifluoromethoxy)-2-(trifluoromethyl)-2H-chromene-3-carboxylic acid, (S)-6,8-dichloro-2-(trifluoromethyl)-2H-1-benzopyran-3-carboxylic acid, (2S)-6-chloro-5,7-dimethyl-2-(trifluoromethyl)-2H-chromene-3-carboxylic acid, prodrugs thereof, salts thereof, isomers thereof, and/or mixtures thereof.

In another embodiment of the invention, the Cox-2 inhibitor can be selected from the class of tricyclic Cox-2 selective inhibitors represented by the general structure of formula II: 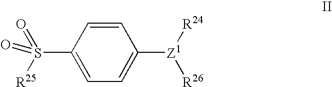
wherein:
Z¹ is selected from the group consisting of partially unsaturated or unsaturated heterocyclyl and partially unsaturated or unsaturated carbocyclic rings;
R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R²⁵ is selected from the group consisting of methyl or amino; and
R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; prodrugs thereof; salts thereof; isomers thereof; and/or mixtures thereof.

In one embodiment of the invention, the tricyclic Cox-2 selective inhibitor comprises at least one compound chosen from celecoxib, parecoxib, deracoxib, valdecoxib, lumiracoxib, etoricoxib, rofecoxib, tilmacoxib, cimicoxib, prodrugs thereof, salts thereof, isomers thereof, and/or mixtures thereof.

In another embodiment of the invention, the Cox-2 selective inhibitor represented by the above Formula II is chosen from those compounds, illustrated in Table 1, which includes celecoxib (B-1), valdecoxib (B-2), deracoxib (B-3), rofecoxib (B-4), etoricoxib (MK-663; B-5), tilmacoxib (JTE-522) (B-6), cimicoxib (B-7), prodrugs thereof, salts thereof, isomers thereof, and/or mixtures thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-9 (U.S. Pat. No. 5,840,924); compound B-10 (WO 00/25779); cimicoxib (4-[4-chloro-5-(3-fluoro-4-methoxyphenyl)-1H-imidzol-1-yl]benzenesulfonamide—CAS RN 265114-23-6); tilmacoxib (4-(4-cyclohexyl-2-methyloxazol-5-yl)-2-fluorobenzenesulfonamide—JTE-522, CAS 180200-68-4); and etoricoxib (CAS RN 202409-33-4, MK-663, SC-86218, and in WO 98/03484).

TABLE 1
Examples of Tricyclic Cox-2 Selective Inhibitors

| Compound | Common | Chemical |
|---|---|---|
| No. | name | name |
| B-1 | celecoxib | 4-[5-(4-methylphenyl)-3-(trifluoromethyl)-1H- |
| | | pyrazol-1-yl] benzenesulfonamide |
| B-2 | valdecoxib | 4-(5-methyl-3-phenyl-4-isoxazolyl) |
| | | benzenesulfonamide |
| B-3 | deracoxib | 4-[3-(difluoromethyl)-5-(3-fluoro-4- |
| | | methoxyphenyl)-1H-pyrazol-1- |
| | | yl]benzenesulfonamide |
| B-4 | rofecoxib | 4-[4-(methylsulfonyl)phenyl]-3-phenyl-2(5H)- |
| | | furanone |
| B-5 | etoricoxib | 2,3′-bipyridine, 5-chloro-6′-methyl-3-[4- |
| | | [methylsulfonyl]phenyl]-; or [2] 5-chloro-6′- |
| | | methyl-3-[p-[methylsulfonyl]phenyl]-2,3′- |
| | | bipyridine |
| B-6 | tilmacoxib | 4-(4-cyclohexyl-2-methyloxazol-5-yl)-2- |
| | | fluorobenzenesulfonamide |
| B-7 | cimicoxib | 4-[4-chloro-5-(3-fluoro-4-methoxyphenyl)-1H- |
| | | imidzol-1-yl]benzenesulfonamide |
| B-8 | parecoxib | N-[[p-(5-methyl-3-phenyl-4-isoxazolyl) |
| | | phenyl]sulfonyl]propionamide |

In yet another embodiment of the invention, the Cox-2 selective inhibitor is selected from the group consisting of celecoxib, valdecoxib, rofecoxib, etoricoxib, tilmacoxib, cimicoxib, etoricoxib, deracoxib, parecoxib, prodrugs thereof, salts thereof, isomers thereof, and/or mixtures thereof. Even more preferred still is that the Cox-2 selective inhibitor is celecoxib.

In another embodiment, the tricyclic Cox-2 selective inhibitor, parecoxib (B-8), N-[[4-(5-methyl-3-phenyl-4-isoxazo-lyl)phenyl]sulfonyl]-; or (2) N-[[p-(5-methyl-3-phenyl-4-isox-azolyl)phenyl]sulfonyl]propionamide; CAS No. 198470-84-7 (See, U.S. Pat. No. 5,932,598), which is a therapeutically effective prodrug of the tricyclic Cox-2 selective inhibitor valdecoxib, compound B-2, (See, U.S. Pat. No. 5,633,272), may be advantageously employed as the Cox-2 inhibitor of the present invention.

A preferred form of parecoxib is sodium parecoxib, which is available as Dynastat®.

Another tricyclic Cox-2 selective inhibitor useful in the present invention is the compound ABT-963, having the structure: 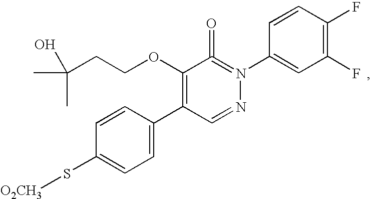
which has been previously described in International Publication Number WO 00/24719.

In a further embodiment of the invention, the Cox-2 inhibitor can be selected from the class of phenylacetic acid derivative Cox-2 selective inhibitors represented by the general structure of formula III: 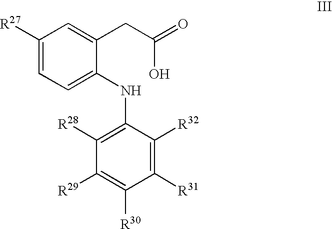
wherein:
R²⁷ is methyl, ethyl, or propyl;
R²⁸ is chloro or fluoro;
R²⁹ is hydrogen, fluoro, or methyl;
R³⁰ is hydrogen, fluoro, chloro, methyl, ethyl, methoxy, ethoxy or hydroxyl;
R³¹ is hydrogen, fluoro, or methyl; and
R³² is chloro, fluoro, trifluoromethyl, methyl, or ethyl, provided that R²⁸, R²⁹, R³⁰ and R³¹ are not all fluoro when R²⁷ is ethyl and R³⁰ is H.

An exemplary phenylacetic acid derivative Cox-2 selective inhibitor that is described in WO 99/11605 is a compound that has the structure shown in formula III,

wherein:

R²⁷ is ethyl;
R²⁸ and R³⁰ are chloro;
R²⁹ and R³¹ are hydrogen; and
R³² is methyl.

Another phenylacetic acid derivative Cox-2 selective inhibitor is a compound that has the structure shown in formula III,

wherein:

R²⁷ is propyl;
R²⁸ and R³⁰ are chloro;
R²⁹ and R³¹ are methyl; and
R³² is ethyl.

Another phenylacetic acid derivative Cox-2 selective inhibitor that is disclosed in WO 02/20090 is a compound that is referred to as COX-189 (also termed lumiracoxib; CAS Reg. No. 220991-20-8), having the structure shown in formula III,

wherein:

R²⁷ is methyl;
R²⁸ is fluoro;
R³² is chloro; and
R²⁹, R³⁰, and R³¹ are hydrogen.

Compounds having a structure similar to that shown in formula VIII, that can serve as the Cox-2 selective inhibitor of the present invention, are described in U.S. Pat. Nos. 6,451,858, 6,310,099, 6,291,523, and 5,958,978.

In certain aspects of the present invention, the Cox-2 selective inhibitor may be a Cox-2 selective inhibitor that is other than any tricyclic Cox-2 selective inhibitor described by formula II. For example, the Cox-2 selective inhibitor may be a chromene Cox-2 inhibitor, which is a class of Cox-2 selective inhibitor that is other than a tricylic Cox-2 selective inhibitor. Likewise, the Cox-2 selective inhibitor may be any compound described by formula III, such as lumiracoxib, which is other than a tricyclic Cox-2 selective inhibitor. Thus, in some embodiments, the present invention encompasses any Cox-2 selective inhibitor that is other than a tricyclic Cox-2 selective inhibitor that is described by formula II, whether such a Cox-2 selective inhibitor is now known or later developed.

In other aspects of the present invention, the Cox-2 selective inhibitor may be at least one compound or class of compounds chosen from Table 2, isomers thereof, salts thereof, and/or mixtures thereof. However, the present invention should not be construed as being limited to any particular one of the Cox-2 selective inhibitors described herein. Indeed, it should be understood that the present invention encompasses any compound that can be shown to act as an inhibitor of the Cox-2 enzyme, whether such a compound is now known, later developed, or even later recognized as having Cox-2 inhibitory activity.

TABLE 2
Additional Cox-2 Selective inhibitors

| | Generic Name/Compound | Trade | | | | |
|---|---|---|---|---|---|---|
| No. | Name | Name(s) | Drug Class/Mode of Action | Dose | Manufacturer | Reference |
| B11 | Nimesulide | | | | | |
| B12 | Flosulide | | | | | |
| B13 | NS-398 | | | | | CAS RN 123653-11-2 |
| | N-(2-cyclohexyloxynitrophenyl) | | | | | Yoshimi, N. et al., in |
| | methane sulfonamide | | | | | Japanese J. Cancer |
| | | | | | | Res., 90(4): 406-412 |
| | | | | | | (1999) |
| B14 | L-745337 | | | | | |
| B15 | RWJ-63556 | | | | | Kirchner et al., in J |
| | | | | | | Pharmacol Exp Ther |
| | | | | | | 282, 1094-1101 |
| | | | | | | (1997) |
| B16 | L-784512 | | | | | |
| B17 | N-(2- | | diarylmethylidenefuran | | | U.S. Pat. No. |
| | cyclohexyloxynitrophenyl)methane | | derivatives | | | 6,180,651 |
| | sulfonamide, and (E)-4- | | | | | |
| | [(4-methylphenyl)(tetrahydro- | | | | | |
| | 2-oxo-3-furanylidene) | | | | | |
| | methyl]benzenesulfonamide | | | | | |
| B18 | Darbufelone | | | | Pfizer | |
| B19 | CS-502 | | | | Sankyo | |
| B20 | LAS 34475 | | | | Almirall | |
| | | | | | Profesfarma | |
| B21 | LAS 34555 | | | | Almirall | |
| | | | | | Profesfarma | |
| B22 | S-33516 | | | | Servier | |
| B23 | SD 8381 | | | | Pharmacia | U.S. Pat. No. |
| | | | | | | 6,034,256 |
| B24 | BMS-347070 | | | | Bristol Myers | U.S. Pat. No. |
| | | | | | Squibb | 6,180,651 |
| B25 | MK-966 | | | | Merck | |
| B26 | L-783003 | | | | Merck | |
| B27 | T-614 | | | | Toyama | |
| B28 | D-1367 | | | | Chiroscience | |
| B29 | L-748731 | | | | Merck | |
| B30 | CT3 | | | | Atlantic | |
| | | | | | Pharmaceutical | |
| B31 | CGP-28238 | | | | Novartis | |
| B32 | BF-389 | | | | Biofor/Scherer | |
| B33 | GR-253035 | | | | Glaxo Wellcome | |
| B34 | 6-dioxo-9H-purin-8-yl-cinnamic | | | | Glaxo Wellcome | |
| | acid | | | | | |
| B35 | S-2474 | | | | Shionogi | |
| B36 | | | Multibinding compounds | | | U.S. Pat. No. |
| | | | containing from 2 to 10 ligands | | | 6,395,724 |
| | | | covanlently attached to one or | | | |
| | | | more linkers | | | |
| B37 | | | Conjugated linoleic acid | | | U.S. Pat. No. |
| | | | derivatives | | | 6,077,868 |
| B38 | | | Heterocyclic aromatic oxazole | | | U.S. Pat. Nos. |
| | | | compounds | | | 5,994,381 |
| | | | | | | and 6,362,209 |
| B39 | | | Miscellaneous compounds | | | U.S. Pat. Nos. |
| | | | | | | 6,596,736, 6,369,275, |
| | | | | | | 6,127,545, 6,130,334, |
| | | | | | | 6,204,387, 6,071,936, |
| | | | | | | 6,001,843 and |
| | | | | | | 6,040,450 |
| B40 | | | Diarylbenzopyran derivatives | | | U.S. Pat. No. |
| | | | | | | 6,340,694 |
| B41 | | | 1-(4-sulfamylaryl)-3-substituted- | | | U.S. Pat. No. |
| | | | 5-aryl-2-pyrazolines | | | 6,376,519 |
| B42 | | | Heterocycle compounds | | | U.S. Pat. No. |
| | | | | | | 6,153,787 |
| B43 | | | 2,3,5-trisubstituted pyridines | | | U.S. Pat. No. |
| | | | | | | 6,046,217 |
| B44 | | | Diaryl bicyclic heterocycles | | | U.S. Pat. No. |
| | | | | | | 6,329,421 |
| B45 | | | Salts of 5-amino or substituted | | | U.S. Pat. No. |
| | | | amino 1,2,3-triazole compounds | | | 6,239,137 |
| B46 | | | Pyrazole derivatives | | | U.S. Pat. No. |
| | | | | | | 6,136,831 |
| B47 | | | Substituted derivatives of | | | U.S. Pat. No. |
| | | | benzosulphonamides | | | |
| B48 | 3-phenyl-4-(4- | | Phenyl heterocycles | | | U.S. Pat. Nos. |
| | (methylsulfonyl)phenyl)-2- | | | | | 5,474,995 and |
| | (2H)-furanone | | | | | 6,239,173 |
| B49 | | | Bicycliccarbonyl indole | | | U.S. Pat. No. |
| | | | compounds | | | 6,303,628 |
| B50 | | | Benzimidazole compounds | | | U.S. Pat. No. |
| | | | | | | 6,310,079 |
| B51 | | | Indole compounds | | | U.S. Pat. No. |
| | | | | | | 6,300,363 |
| B52 | | | Aryl phenylhydrazides | | | U.S. Pat. No. |
| | | | | | | 6,077,869 |
| B53 | | | 2-aryloxy, 4-aryl furan-2-ones | | | U.S. Pat. No. |
| | | | | | | 6,140,515 |
| B54 | | | Bisaryl compounds | | | U.S. Pat. No. |
| | | | | | | 5,994,379 |
| B55 | | | 1,5-diarylpyrazoles | | | U.S. Pat. No. |
| | | | | | | 6,028,202 |
| B56 | | | 2-substituted imidazoles | | | U.S. Pat. No. |
| | | | | | | 6,040,320 |
| B57 | | | 1,3- and 2,3-diarylcycloalkano | | | U.S. Pat. No. |
| | | | and cycloalkeno pyrazoles | | | 6,083,969 |
| B58 | | | Esters derived from | | | U.S. Pat. No. |
| | | | indolealkanols and novel | | | 6,306,890 |
| | | | amides derived from | | | |
| | | | indolealkylamides | | | |
| B59 | | | Pyridazinone compounds | | | U.S. Pat. No. |
| | | | | | | 6,307,047 |
| B60 | | | Benzosulphonamide derivatives | | | U.S. Pat. No. |
| | | | | | | 6,004,948 |
| B61 | | | Methanesulfonyl-biphenyl | | | U.S. Pat. No. |
| | | | derivatives | | | 6,583,321 |
| B62 | | | 1H-indole derivatives | | | U.S. Pat. No. |
| | | | | | | 6,599,929 |
| B63 | N-(2-hydroxyethyl)-4-[5-(4- | | Certain prodrugs of Cox-2 | | | U.S. Pat. Nos. |
| | methylphenyl)-3- | | inhibitors | | | 6,436,967 and |
| | (trifluoromethyl)-1H-pyrazol-1- | | | | | 6,613,790 |
| | yl]benzenesulfonamide, N,N- | | | | | |
| | bis(2-hydroxyethyl)-4-[5-(4- | | | | | |
| | methylphenyl)-3- | | | | | |
| | (trifluoromethyl)-1H-pyrazol-1- | | | | | |
| | yl]benzenesulfonamide | | | | | |
| B64 | | | Sulfamoylheteroaryl pyrazole | | | U.S. Pat. No. |
| | | | compounds | | | 6,583,321 |
| B65 | | | Heteroaryl substituted amidinyl | | | U.S. Pat. No. |
| | | | and imidazolyl compounds | | | 6,555,563 |
| B66 | | | Substituted hydroxamic acid | | | U.S. Pat. Nos. |
| | | | derivatives | | | 6,432,999, 6,512,121, |
| | | | | | | and 6,515,014 |
| B67 | | | Pyrazolopyridine compounds | | | U.S. Pat. No. |
| | | | | | | 6,498,166 |
| B68 | | | 4,5-diaryl-3(2H)-furanone | | | U.S. Pat. No. |
| | | | derivatives | | | 6,492,416 |
| B69 | | | 2-phenyl-1,2-benzisoselenazol- | | | U.S. Pat. No. |
| | | | 3(2H)-one derivatives and 2- | | | 6,492,416 |
| | | | phenylcarbomyl-phenylselenyl | | | |
| | | | derivatives | | | |
| B70 | | | Pyrones | | | U.S. Pat. No. |
| | | | | | | 6,465,509 |
| B71 | | | Organically synthesized or | | | U.S. Published |
| | | | purified from plant sources, free- | | | Application No. |
| | | | B-ring flavanoids | | | 2003/0165588 |
| B72 | | | Heterocyclo-alkylsulfonyl | | | European Patent |
| | | | pyrazoles | | | Application No. EP |
| | | | | | | 1312367 |
| B73 | | | 2-phenylpyran-4-one derivatives | | | U.S. Pat. No. |
| | | | | | | 6,518,303 |
| B74 | | | Sulfonylphenylpyrazoles | | | U.S. Pat. No. |
| | | | | | | 6,472,416 |
| B75 | | | 2,3-diaryl-pyrazolo[1,5- | | | U.S. Pat. No. |
| | | | b]pyridazines | | | 6,451,794 |
| B76 | | | (Methylsulfonyl)phenyl | | | U.S. Pat. Nos. |
| | | | furanones | | | 6,169,188, 6,020,343, |
| | | | | | | and 5,981,576 |
| B77 | | | Diaryl-2-(5H)-furanones | | | U.S. Pat. No. |
| | | | | | | 6,222,048 |
| B78 | | | 3,4-diaryl-2-hydroxy-2,5- | | | U.S. Pat. No. |
| | | | dihydrofurans | | | 6,057,319 |
| B79 | | | Carbocyclic sulfonamides | | | U.S. Pat. No. |
| | | | | | | 6,046,236 |
| B80 | | | Oxazole derivatives | | | U.S. Pat. Nos. |
| | | | | | | 6,002,014 and |
| | | | | | | 5,945,539 |
| B81 | | | C-nitroso compounds | | | U.S. Pat. Nos. |
| | | | | | | 6,359,182 and |
| | | | | | | 6,538,116 |
| B82 | | | Substituted pyridines | | | U.S. Published |
| | | | | | | Application No. |
| | | | | | | 2003/0065011 |
| B83 | | | Substituted indole derivatives | | | U.S. Published |
| | | | | | | Application No. |
| | | | | | | 2003/0207897 |
| B84 | meloxicam | | | | | CAS registry number |
| | | | | | | 71125-38-7 |
| B85 | RS 57067 | | | | | CAS registry number |
| | 6-[[5-(4-chlorobenzoyl)-1,4- | | | | | 179382-91-3 |
| | dimethyl-1H-pyrrol-2- | | | | | |
| | yl]methyl]-3(2H)- | | | | | |
| | pyridazinone | | | | | |

Examples of specific compounds that are useful as Cox-2 selective inhibitors include, without limitation:
8-acetyl-3-(4-fluorophenyl)-2-(4-methylsulfonyl)phenyl-imidazo(1,2-a)pyridine; 5,5-dimethyl-4-(4-methylsulfonyl)phenyl-3-phenyl-2-(5H)-furanone; 5-(4-fluorophenyl)-1-[4-(methylsulfonyl)phenyl]-3-(trifluoromethyl)pyrazole; 4-(4-fluorophenyl)-5-[4-(methylsulfonyl)phenyl]-1-phenyl-3-(trifluoromethyl)pyrazole; 4-(5-(4-chlorophenyl)-3-(4-methoxyphenyl)-1H-pyrazol-1-yl)benzenesulfonamide; 4-(3,5-bis(4-methylphenyl)-1H-pyrazol-1-yl)benzenesulfonamide; 4-(5-(4-chlorophenyl)-3-phenyl-1H-pyrazol-1-yl)benzenesulfonamide; 4-(3,5-bis(4-methoxyphenyl)-1H-pyrazol-1-yl)benzenesulfonamide; 4-(5-(4-chlorophenyl)-3-(4-methylphenyl)-1H-pyrazol-1-yl)benzenesulfonamide; 4-(5-(4-chlorophenyl)-3-(4-nitrophenyl)-1H-pyrazol-1-yl)benzenesulfonamide; 4-(5-(4-chlorophenyl)-3-(5-chloro-2-thienyl)-1H-pyrazol-1-yl)benzenesulfonamide; 4-(4-chloro-3,5-diphenyl-1H-pyrazol-1-yl)benzenesulfonamide; 4-[5-(4-chlorophenyl)-3-(trifluoromethyl)-1H-pyrazol-1-yl]benzenesulfonamide; 4-[5-phenyl-3-(trifluoromethyl)-1H-pyrazol-1-yl]benzenesulfonamide; 4-[5-(4-fluorophenyl)-3-(trifluoromethyl)-1H-pyrazol-1-yl]benzenesulfonamide; 4-[5-(4-methoxyphenyl)-3-(trifluoromethyl)-1H-pyrazol-1-yl]benzenesulfonamide; 4-[5-(4-chlorophenyl)-3-(difluoromethyl)-1H-pyrazol-1-yl]benzenesulfonamide; 4-[5-(4-methylphenyl)-3-(trifluoromethyl)-1H-pyrazol-1-yl]benzenesulfonamide; 4-[4-chloro-5-(4-chlorophenyl)-3-(trifluoromethyl)-1H-pyrazol-1-yl]benzenesulfonamide; 4-[3-(difluoromethyl)-5-(4-methylphenyl)-1H-pyrazol-1-yl]benzenesulfonamide; 4-[3-(difluoromethyl)-5-phenyl-1H-pyrazol-1-yl]benzenesulfonamide; 4-[3-(difluoromethyl)-5-(4-methoxyphenyl)-1H-pyrazol-1-yl]benzenesulfonamide; 4-[3-cyano-5-(4-fluorophenyl)-1H-pyrazol-1-yl]benzenesulfonamide; 4-[3-(difluoromethyl)-5-(3-fluoro-4-methoxyphenyl)-1H-pyrazol-1-yl]benzenesulfonamide; 4-[5-(3-fluoro-4-methoxyphenyl)-3-(trifluoromethyl)-1H-pyrazol-1-yl]benzenesulfonamide; 4-[4-chloro-5-phenyl-1H-pyrazol-1-yl]benzenesulfonamide; 4-[5-( 4-chlorophenyl)-3-(hydroxymethyl)-1H-pyrazol-1-yl]benzenesulfonamide; 4-[5-(4-(N,N-dimethylamino)phenyl)-3-(trifluoromethyl)-1H-pyrazol-1-yl]benzenesulfonamide; 5-(4-fluorophenyl)-6-[4-(methylsulfonyl)phenyl]spiro[2.4]hept-5-ene; 4-[6-(4-fluorophenyl)spiro[2.4]hept-5-en-5-yl]benzenesulfonamide; 6-(4-fluorophenyl)-7-[4-(methylsulfonyl)phenyl]spiro[3.4]oct-6-ene; 5-(3-chloro-4-methoxyphenyl)-6-[4-(methylsulfonyl)phenyl]spiro[2.4]hept-5-ene; 4-[6-(3-chloro-4-methoxyphenyl)spiro[2.4]hept-5-en-5-yl]benzenesulfonamide; 5-(3,5-dichloro-4-methoxyphenyl)-6-[4-(methylsulfonyl)phenyl]spiro[2.4]hept-5-ene; 5-(3-chloro-4-fluorophenyl)-6-[4-(methylsulfonyl)phenyl]spiro[2.4]hept-5-ene; 4-[6-(3,4-dichlorophenyl)spiro[2.4]hept-5-en-5-yl]benzenesulfonamide; 2-(3-chloro-4-fluorophenyl)-4-(4-fluorophenyl)-5-(4-methylsulfonylphenyl)thiazole; 2-(2-chlorophenyl)-4-(4-fluorophenyl)-5-(4-methylsulfonylphenyl)thiazole; 5-(4-fluorophenyl)-4-(4-methylsulfonylphenyl)-2-methylthiazole; 4-(4-fluorophenyl)-5-(4-methylsulfonylphenyl)-2-trifluoromethylthiazole; 4-(4-fluorophenyl)-5-(4-methylsulfonylphenyl)-2-(2-thienyl)thiazole; 4-(4-fluorophenyl)-5-(4-methylsulfonylphenyl)-2-benzylaminothiazole; 4-(4-fluorophenyl)-5-(4-methylsulfonylphenyl)-2-(1-propylamino)thiazole; 2-[(3,5-dichlorophenoxy)methyl)-4-(4-fluorophenyl)-5-[4-(methylsulfonyl)phenyl]thiazole; 5-(4-fluorophenyl)-4-(4-methylsulfonylphenyl)-2-trifluoromethylthiazole; 1-methylsulfonyl-4-[1,1-dimethyl-4-(4-fluorophenyl)cyclopenta-2,4-dien-3-yl]benzene; 4-[4-(4-fluorophenyl)-1,1-dimethylcyclopenta-2,4-dien-3-yl]benzenesulfonamide; 5-(4-fluorophenyl)-6-[4-(methylsulfonyl)phenyl]spiro[2.4]hepta-4,6-diene; 4-[6-(4-fluorophenyl)spiro[2.4]hepta-4,6-dien-5-yl]benzenesulfonamide; 6-(4-fluorophenyl)-2-methoxy-5-[4-(methylsulfonyl)phenyl]-pyridine-3-carbonitrile; 2-bromo-6-(4-fluorophenyl)-5-[4-(methylsulfonyl)phenyl]-pyridine-3-carbonitrile; 6-(4-fluorophenyl)-5-[4-(methylsulfonyl)phenyl]-2-phenyl-pyridine-3-carbonitrile; 4-[2-(4-methylpyridin-2-yl)-4-(trifluoromethyl)-1H-imidazol-1-yl]benzenesulfonamide; 4-[2-(5-methylpyridin-3-yl)-4-(trifluoromethyl)-1H-imidazol-1-yl]benzenesulfonamide; 4-[2-(2-methylpyridin-3-yl)-4-(trifluoromethyl)-1H-imidazol-1-yl]benzenesulfonamide; 3-[1-[4-(methylsulfonyl)phenyl]-4-(trifluoromethyl)-1H-imidazol-2-yl]pyridine; 2-[1-[4-(methylsulfonyl)phenyl-4-(trifluoromethyl)-1H-imidazol-2-yl]pyridine; 2-methyl-4-[1-[4-(methylsulfonyl)phenyl-4-(trifluoromethyl)-1H-imidazol-2-yl]pyridine; 2-methyl-6-[1-[4-(methylsulfonyl)phenyl-4-(trifluoromethyl)-1H-imidazol-2-yl]pyridine; 4-[2-(6-methylpyridin-3-yl)-4-(trifluoromethyl)-1H-imidazol-1-yl]benzenesulfonamide; 2-(3,4-difluorophenyl)-1-[4-(methylsulfonyl)phenyl]-4-(trifluoromethyl)-1H-imidazole; 4-[2-(4-methylphenyl)-4-(trifluoromethyl)-1H-imidazol-1-yl]benzenesulfonamide; 2-(4-chlorophenyl)-1-[4-(methylsulfonyl)phenyl]-4-methyl-1H-imidazole; 2-(4-chlorophenyl)-1-[4-(methylsulfonyl)phenyl]-4-phenyl-1H-imidazole; 2-(4-chlorophenyl)-4-(4-fluorophenyl)-1-[4-(methylsulfonyl)phenyl]-1H-imidazole; 2-(3-fluoro-4-methoxyphenyl)-1-[4-(methylsulfonyl)phenyl-4-(trifluoromethyl)-1H-imidazole; 1-[4-(methylsulfonyl)phenyl]-2-phenyl-4-trifluoromethyl-1H-imidazole; 2-(4-methylphenyl)-1-[4-(methylsulfonyl)phenyl]-4-trifluoromethyl-1H-imidazole; 4-[2-(3-chloro-4-methylphenyl)-4-(trifluoromethyl)-1H-imidazol-1-yl]benzenesulfonamide; 2-(3-fluoro-5-methylphenyl)-1-[4-(methylsulfonyl)phenyl]-4-(trifluoromethyl)-1H-imidazole; 4-[2-(3-fluoro-5-methylphenyl)-4-(trifluoromethyl)-1H-imidazol-1-yl]benzenesulfonamide; 2-(3-methylphenyl)-1-[4-(methylsulfonyl)phenyl]-4-trifluoromethyl-1H-imidazole; 4-[2-(3-methylphenyl)-4-trifluoromethyl-1H-imidazol-1-yl]benzenesulfonamide; 1-[4-(methylsulfonyl)phenyl]-2-(3-chlorophenyl)-4-trifluoromethyl-1H-imidazole; 4-[2-(3-chlorophenyl)-4-trifluoromethyl-1H-imidazol-1-yl]benzenesulfonamide; 4-[2-phenyl-4-trifluoromethyl-1H-imidazol-1-yl]benzenesulfonamide; 4-[2-(4-methoxy-3-chlorophenyl)-4-trifluoromethyl-1H-imidazol-1-yl]benzenesulfonamide; 1-allyl-4-(4-fluorophenyl)-3-[4-(methylsulfonyl)phenyl]-5-(trifluoromethyl)-1H-pyrazole; 4-[1-ethyl-4-(4-fluorophenyl)-5-(trifluoromethyl)-1H-pyrazol-3-yl]benzenesulfonamide; N-phenyl-[4-(4-luorophenyl)-3-[4-(methylsulfonyl)phenyl]-5-(trifluoromethyl)-1H-pyrazol-1-yl]acetamide; ethyl [4-(4-fluorophenyl)-3-[4-(methylsulfonyl)phenyl]-5-(trifluoromethyl)-1H-pyrazol-1-yl]acetate; 4-(4-fluorophenyl)-3-[4-(methylsulfonyl)phenyl]-1-(2-phenylethyl)-1H-pyrazole; 4-(4-fluorophenyl)-3-[4-(methylsulfonyl)phenyl]-1-(2-phenylethyl)-5-(trifluoromethyl)pyrazole; 1-ethyl-4-(4-fluorophenyl)-3-[4-(methylsulfonyl)phenyl]-5-(trifluoromethyl)-1H-pyrazole; 5-(4-fluorophenyl)-4-(4-methylsulfonylphenyl)-2-trifluoromethyl-1H-imidazole; 4-[4-(methylsulfonyl)phenyl]-5-(2-thiophenyl)-2-(trifluoromethyl)-1H-imidazole; 5-(4-fluorophenyl)-2-methoxy-4-[4-(methylsulfonyl)phenyl]-6-(trifluoromethyl)pyridine; 2-ethoxy-5-(4-fluorophenyl)-4-[4-(methylsulfonyl)phenyl]-6-(trifluoromethyl)pyridine; 5-(4-fluorophenyl)-4-[4-(methylsulfonyl)phenyl]-2-(2-propynyloxy)-6-(trifluoromethyl)pyridine; 2-bromo-5-(4-fluorophenyl)-4-[4-(methylsulfonyl)phenyl]-6-(trifluoromethyl)pyridine; 4-[2-(3-chloro-4-methoxyphenyl)-4,5-difluorophenyl]benzenesulfonamide; 1-(4-fluorophenyl)-2-[4-(methylsulfonyl)phenyl]benzene; 5-difluoromethyl-4-(4-methylsulfonylphenyl)-3-phenylisoxazole; 4-[3-ethyl-5-phenylisoxazol-4-yl]benzenesulfonamide; 4-[5-difluoromethyl-3-phenylisoxazol-4-yl]benzenesulfonamide; 4-[5-hydroxymethyl-3-phenylisoxazol-4-yl]benzenesulfonamide; 4-[5-methyl-3-phenyl-isoxazol-4-yl]benzenesulfonamide; 1-[2-(4-fluorophenyl)cyclopenten-1-yl]-4-(methylsulfonyl)benzene; 1-[2-(4-fluoro-2-methylphenyl)cyclopenten-1-yl]-4-(methylsulfonyl)benzene; 1-[2-(4-chlorophenyl)cyclopenten-1-yl]-4-(methylsulfonyl)benzene; 1-[2-(2,4-dichlorophenyl)cyclopenten-1-yl]-4-(methylsulfonyl)benzene; 1-[2-(4-trifluoromethylphenyl)cyclopenten-1-yl]-4-(methylsulfonyl)benzene; 1-[2-(4-methylthiophenyl)cyclopenten-1-yl]-4-(methylsulfonyl)benzene; 1-[2-(4-fluorophenyl)-4,4-dimethylcyclopenten-1-yl]-4-(methylsulfonyl)benzene; 4-[2-(4-fluorophenyl)-4,4-dimethylcyclopenten-1-yl]benzenesulfonamide; 1-[2-(4-chlorophenyl)-4,4-dimethylcyclopenten-1-yl]-4-(methylsulfonyl)benzene; 4-[2-(4-chlorophenyl)-4,4-dimethylcyclopenten-1-yl]benzenesulfonamide; 4-[2-(4-fluorophenyl)cyclopenten-1-yl]benzenesulfonamide; 4-[2-(4-chlorophenyl)cyclopenten-1-yl]benzenesulfonamide; 1-[2-(4-methoxyphenyl)cyclopenten-1-yl]-4-(methylsulfonyl)benzene; 1-[2-(2,3-difluorophenyl)cyclopenten-1-yl]-4-(methylsulfonyl)benzene; 4-[2-(3-fluoro-4-methoxyphenyl)cyclopenten-1-yl]benzenesulfonamide; 1-[2-(3-chloro-4-methoxyphenyl)cyclopenten-1-yl]-4-(methylsulfonyl)benzene; 4-[2-(3-chloro-4-fluorophenyl)cyclopenten-1-yl]benzenesulfonamide; 4-[2-(2-methylpyridin-5-yl)cyclopenten-1-yl]benzenesulfonamide; ethyl 2-[4-(4-fluorophenyl)-5-[4-(methylsulfonyl)phenyl]oxazol-2-yl]-2-benzyl-acetate; 2-[4-(4-fluorophenyl)-5-[4-(methylsulfonyl)phenyl]oxazol-2-yl]acetic acid; 2-(tert-butyl)-4-(4-fluorophenyl)-5-[4-(methylsulfonyl)phenyl]oxazole; 4-(4-fluorophenyl)-5-[4-(methylsulfonyl)phenyl]-2-phenyloxazole; 4-(4-fluorophenyl)-2-methyl-5-[4-(methylsulfonyl)phenyl]oxazole; 4-[5-(3-fluoro-4-methoxyphenyl)-2-trifluoromethyl-4-oxazolyl]benzenesulfonamide; salts thereof, isomers thereof, and/or mixtures thereof.

Cox-2 inhibitors that are useful in the methods and compositions of present invention can be supplied by any source as long as the Cox-2 inhibitor is pharmaceutically acceptable. Likewise, Cox-2 inhibitors that are useful in the compositions and methods of present invention can be synthesized, for example, according to the description in Example 1. Several Cox-2 inhibitors that are suitable for use with the compositions and methods of the present invention may be synthesized by the methods described in, for example, in U.S. Pat. No. 5,466,823 to Talley, et al.

Various classes of Cox-2 inhibitors useful in the present invention can be prepared as follows. Pyrazoles can be prepared by methods described in WO 95/15316. Pyrazoles can further be prepared by methods described in WO 95/15315. Pyrazoles can also be prepared by methods described in WO 96/03385. Thiophene analogs useful in the present invention can be prepared by methods described in WO 95/00501. Preparation of thiophene analogs is also described in WO 94/15932. Oxazoles useful in the present invention can be prepared by the methods described in WO 95/00501. Preparation of oxazoles is also described in WO 94/27980. Isoxazoles useful in the present invention can be prepared by the methods described in WO 96/25405. Imidazoles useful in the present invention can be prepared by the methods described in WO 96/03388. Preparation of imidazoles is also described in WO 96/03387. Cyclopentene Cox-2 inhibitors useful in the present invention can be prepared by the methods described in U.S. Pat. No. 5,344,991. Preparation of cyclopentene Cox-2 inhibitors is also described in WO 95/00501. Terphenyl compounds useful in the present invention can be prepared by the methods described in WO 96/16934. Thiazole compounds useful in the present invention can be prepared by the methods described in WO 96/03,392. Pyridine compounds useful in the present invention can be prepared by the methods described in WO 96/03392. The preparation of pyridine compounds is also described in WO 96/24,585. Benzopyranopyrazolyl compounds useful in the present invention can be prepared by the methods described in WO 96/09304. Chromene compounds useful in the present invention can be prepared by the methods described in WO 98/47890. Preparation of chromene compounds is also described in WO 00/23433. Chromene compounds can further be prepared by the methods described in U.S. Pat. No. 6,077,850. Preparation of chromene compounds is further described in U.S. Pat. No. 6,034,256. Arylpyridazinones useful in the present invention can be prepared by the methods described in WO 00/24719. Preparation of arylpyridazinones is also described in WO 99/10332. Arylpyridazinones can further be prepared by the methods described in WO 99/10331. 5-Alkyl-2-arylaminophenylacetic acids and derivatives useful in the present invention can be prepared by the methods described in WO 99/11605. Diarylmethylidenefuran derivative Cox-2 selective inhibitors useful in the present invention can be prepared by the methods described in U.S. Pat. No. 6,180,651. The celecoxib used in the compositions and methods of the present invention can be prepared in the manner set forth in U.S. Pat. No. 5,466,823. The valdecoxib used in the compositions and methods of the present invention can be prepared in the manner set forth in U.S. Pat. No. 5,633,272. The parecoxib used in the compositions and methods of the present invention can be prepared in the manner set forth in U.S. Pat. No. 5,932,598. The rofecoxib used in the compositions and methods of the present invention can be prepared in the manner set forth in U.S. Pat. No. 5,474,995. The deracoxib used in the compositions and methods of the present invention can be prepared in the manner set forth in U.S. Pat. No. 5,521,207. The etoricoxib used in the compositions and methods of the present invention can be prepared in the manner set forth in WO 98/03484. The cimicoxib used in the compositions and methods of the present invention can be prepared in the manner set forth in Drugs of the Future, 29(4):325-330 (2004). The meloxicam used in the compositions and methods of the present invention can be prepared in the manner set forth in U.S. Pat. No. 4,233,299. The compound 4-(4-cyclohexyl-2-methyloxazol-5-yl)-2-fluorobenzenesulfonamide used in the compositions and methods of the present invention can be prepared in the manner set forth in U.S. Pat. No. 5,994,381. The compound 2-(3,4-difluorophenyl)-4-(3-hydroxy-3-methylbutoxy)-5-[4-(methylsulfonyl)phenyl]-3(2H)-pyridazinone used in the compositions and methods of the present invention can be prepared in the manner set forth in WO 00/24719. The compound 2-(3,5-difluorophenyl)-3-[4-(methylsulfonyl)phenyl]-2-cyclopenten-1-one used in the compositions and methods of the present invention can be prepared in the manner set forth in EP 863134. The compound 2-[(2-chloro-6-fluorophenyl)amino]-5-methyl-benzeneacetic acid used in the compositions and methods of the present invention can be prepared in the manner set forth in WO 99/11605. The compound N-[2-(cyclohexyloxy)-4-nitrophenyl]methanesulfonamide used in the compositions and methods of the present invention can be prepared in the manner set forth in U.S. Pat. No. 4,885,367. The compound (3Z)-3-[(4-chlorophenyl)[4-(methylsulfonyl)phenyl]methylene]dihydro-2(3H)-furanone used in the compositions and methods of the present invention can be prepared in the manner set forth in U.S. Pat. No. 6,180,651.

Cox-2 inhibitors can also be isolated and purified from natural sources. Cox-2 inhibitors should be of a quality and purity that is conventional in the trade for use in pharmaceutical products.

The present invention is also directed to a novel method of preventing or treating bone disorders and bone disorder-related complications in a subject comprising administering to the subject a Cox-2 inhibitor in combination with one or more bone disorder treatment agents.

As used herein, the term “bone disorder treatment agent” refers to any chemical recognized as having an effect on bone disorders or a bone disorder agent, whether in vivo or in vitro, over any duration of time other than a chemical that is an inhibitor of the Cox-2 enzyme. This effect can occur via the reduction of the rate of bone loss, increase in the rate of bone formation, inflammation reduction, pain reduction, or by any other mechanism.

Although any combination of a Cox-2 inhibitor and bone disorder treatment agent is encompassed by the present invention, preferred examples of bone disorder treatment agents include those agents specifically recited in table 3 and elsewhere herein or isomers thereof, salts thereof, and/or mixtures thereof. However, the present invention should not be construed as being limited to any particular one of the Cox-2 inhibitors or bone disorder treatment agents described herein. Thus, it is further preferred that any bone disorder treatment agent recited in table 3 be combined in methods, compositions, pharmaceutical compositions, and kits with any inhibitor of the Cox-2 enzyme. More preferred still are combinations of Cox-2 inhibitors with one or more bone disorder treatment agents. Even further preferred are methods, compositions, pharmaceutical compositions, and kits comprising Cox-2 inhibitors alone and in combination with one or more bone disorder treatment agents. It should also be understood that the present invention encompasses any bone disorder treatment agent that can be shown to act as a bone disorder treatment agent, whether such a compound is now known, later developed, or even later recognized as having efficacy for treating or preventing a bone disorder, as long as the compound is other than a Cox-2 inhibitor.

TABLE 3
Bone Disorder Treatment Agents

| | | Trade | Drug | | | |
|---|---|---|---|---|---|---|
| No. | Compound Name | Name(s) | Class | Dose | Manufacturer | Reference |
| A1 | Risedronate | Actonel ® | Bisphosphonate - | 5 mg tablet, once a | Proctor & Gamble | Physician's Desk |
| | [1-hydroxy-2-(3- | | Antiresorptive | day | Pharmaceuticals | Reference, 55^{th} Edition |
| | pyridinyl)ethyliden]bis[phosphonic | | Agent | | | (2001). |
| | acid] | | | | | |
| A2 | Alendronate | Fosamax ® | Bisphosphonate - | 5 mg tablet, once a | Merck & Co., Inc. | Physician's Desk |
| | 4-amino-1- | | Antiresorptive | day | | Reference, 55^{th} Edition |
| | hydroxybulylidene | | Agent | | | (2001). |
| | bisphophonic acid | | | | | |
| A3 | Calcitonin | Miacalcin ®; | Polypeptide | 100 I.U. injected | Novartis | Physician's Desk |
| | 32-amino-acid | Calcimar ® | | once every other | Consumer Health, | Reference, 55^{th} Edition |
| | polypeptide | | | day; or 200 I.U. | Inc. | (2001). |
| | | | | daily administerered | | |
| | | | | intranasally, | | |
| | | | | alternating nostrils | | |
| | | | | daily. | | |
| A4 | Etidronate | Didronel ® | Bisphosphonate | 5-20 mg/kg/day | Proctor & Gamble | Physician's Desk |
| | (1- | | | | Pharmaceuticals | Reference, 55^{th} Edition |
| | hydroxyethylidene)diphosphonic | | | | | (2001). |
| | acid | | | | | |
| A5 | Raloxifene | Evista ® | Selective | 60 mg tablet taken | Eli Lilly and | Physician's Desk |
| | Methanone, [6-hydroxy- | | estrogen | orally once a day | Company | Reference, 55^{th} Edition |
| | 2-(4- | | receptor | | | (2001). |
| | hydroxyphenyl)benzo[b]thien- | | modulator | | | |
| | 3-yl] - [4-[2-(1- | | | | | |
| | piperidinyl)ethoxy]phenyl]-, | | | | | |
| | hydrochloride | | | | | |
| A6 | Estradiol | Gynodiol ™; | Estrogen | 0.5-2.0 mg tablet; | Fielding | Physician's Desk |
| | Estra-1,3,5(10)-triene- | Climara ®; | | 0.025-0.1 mg per | Pharmaceutical | Reference, 55^{th} Edition |
| | 3,17β-diol | Alora ®; | | day, transdermal | Company; Berlex | (2001). |
| | | Activella ™ | | patch | Laboratories; | |
| | | | | | Watson Pharma, | |
| | | | | | Inc; Pharmacia | |
| A7 | Esterified Estrogen | Estratab ®; | Estrogen | 0.3-1.25 mg per day | Solvay | Physician's Desk |
| | | Menest ® | | | Pharmaceuticals, | Reference, 55^{th} Edition |
| | | | | | Inc.; Monarch | (2001). |
| | | | | | Pharmaceuticals, | |
| A8 | Estropipate (Piperazine | Ogen ®; | Estrogen | 0.625 mg tablet, | Pharmacia & | Physician's Desk |
| | Estrone Sulphate) | Ortho-Est ® | | once daily | Upjohn Compnay | Reference, 55^{th} Edition |
| | Estra-1,3,5(10)-trien-17- | | | | | (2001). |
| | one,3-(sulfooxy)-with | | | | | |
| | piperazine (1:1) | | | | | |
| A9 | Ethinyl Estradiol | Estinyl ® | Estrogen | 0.02-0.05 mg tablet, | Schering | Physician's Desk |
| | 19-Nor-17α-pregna- | | | once daily | Corporation | Reference, 55^{th} Edition |
| | 1,3,5(10)-trien-20-yne- | | | | | (2001). |
| | 3,17-diol | | | | | |
| A10 | Conjugated Estrogen | Premphase ®; | Estrogen | 1 tablet taken orally, | Wyeth-Ayerst | Physician's Desk |
| | and | Prempro ™ | | once a day. Each | Company | Reference, 55^{th} Edition |
| | Medroxyprogesterone | | | tablet contains | | (2001). |
| | Acetate (MPA) | | | 0.625 mg | | |
| | MPA: pregn-4-ene-3,20- | | | conjugated | | |
| | dione, 17-(acetyloxy)-6- | | | estrogen and either | | |
| | methyl-,(6α) | | | 2.5 or 5 mg MPA | | |
| A11 | Conjugated Estrogen | Premarin ®; | Estrogen | 0.625 mg/d, one | Wyeth-Averst | Physician's Desk |
| | | Prempro ®; | | tablet per day | Company | Reference, 55^{th} Edition |
| | | Endometrion | | | | (2001). |
| A12 | Teriparatide | Fortéo ® | Parathyroid | 20 μg, injected once | Eli Lilly and | |
| | | | Hormone | daily | Company | |
| A13 | Calcium | Citracal ®; | Vitamin | 1.5 g total per day | R&D Laboratories, | Physician's Desk |
| | | Calci-chew ® | | (food + supplement) | Inc. | Reference, 55^{th} Edition |
| | | | | | | (2001). |
| A14 | Vitamin D | Citracal ® | Vitamin | 400 IU/day | R&D Laboratories, | Physician's Desk |
| | 1,25-dihydroxyvitamin D | Caplets + D; | | | Inc. | Reference, 55^{th} Edition |
| | | Citracal ® | | | | (2001); US Patent |
| | | Plus; | | | | 6,544,969 |
| A15 | Pamidronate | Aredia ® | Bisphosphonate | 30 mg diluted in | Novartis | Physician's Desk |
| | Phosphonic acid (3- | | | 500 mL Sodium | Consumer Health, | Reference, 55^{th} Edition |
| | amino-1- | | | Chloride, by | Inc. | (2001). |
| | hydroxypropylidene) bis-, | | | intravenous over a | | |
| | disodium salt | | | 4 hour period for 3 | | |
| | | | | days. | | |
| A16 | Tiludronate | Skelid ® | Bisphosphonate | 400 mg daily, taken | Sanofi-Synthelabo | Physician's Desk |
| | [[(4-Chlorophenyl) | | | orally, for 3 months. | inc. | Reference, 55^{th} Edition |
| | thio]methylene]bis[phosphonic | | | | | (2001). |
| | acid], disodium | | | | | |
| | salt | | | | | |
| A17 | 4-aminobutanoic acid | | | | | U.S. Pat. No. 6,569,899 |
| | derivatives | | | | | |
| A18 | Thienyl substituted | | | | | U.S. Pat. No. 6,566,366 |
| | acylguanidines | | | | | |
| A19 | | | Osteoclast- | | | U.S. Pat. No. 6,566,366 |
| | | | secreted | | | |
| | | | proteins | | | |
| A20 | | | Growth | | | U.S. Pat. No. 6,559,150 |
| | | | Hormone | | | |
| | | | Secretagogues | | | |
| A21 | | | Integrin | | | U.S. Pat. No. 6,559,144 |
| | | | Inhibitors | | | |
| A22 | | | Interleukin-6 | | | U.S. Pat. No. 6,555,555 |
| | | | Inhibitor | | | |
| A23 | | | Interleukin-12 | | | U.S. Pat. No. 6,555,555 |
| | | | Inhibitor | | | |
| A24 | | | Matrix | | | U.S. Pat. No. 6,541,521 |
| | | | Metalloproteinase | | | |
| | | | Inhibitors | | | |
| A25 | Genus Anethum Extract | | | | | U.S. Pat. No. 6,534,098 |
| A26 | | | Phosphodiesterase | | | U.S. Pat. No. 6,531,498 |
| | | | VII inhibitors | | | |
| A27 | | | Glycosylated | | | U.S. Pat. No. 6,531,454 |
| | | | Polyamines | | | |
| A28 | Halogenated | | Selective | | | U.S. Pat. No. 6,528,681 |
| | triphenylethylene | | estrogen | | | |
| | derivatives | | receptor | | | |
| | | | modulator | | | |
| A29 | | | Pyridine | | | U.S. Pat. No. 6,521,643 |
| | | | compounds | | | |
| A30 | | | Cysteine | | | U.S. Pat. No. 6,506,733 |
| | | | protease | | | |
| | | | inhibitors | | | |
| A31 | | | Prostaglandin | | | U.S. Pat. No. 6,498,172 |
| | | | agonists | | | |
| A32 | Condensed 4,5,6,7- | | | | | U.S. Pat. No. 6,489,351 |
| | tetrahydrobenzo[C]thiophene | | | | | |
| A33 | Arylalkanoylpyridazine | | | | | U.S. Pat. No. 6,479,494 |
| A34 | Disulfide | | | | | U.S. Pat. No. 6,468,993 |
| A35 | | | Thiol-containing | | | U.S. Pat. No. 6,468,993 |
| | | | compounds | | | |
| A36 | Beta-amino acid nitrile | | | | | U.S. Pat. No. 6,462,076 |
| A37 | | | Amino-alcohol | | | U.S. Pat. No. 6,444,832 |
| | | | derivatives | | | |
| A38 | Androst-5-ene- | | | | | U.S. Pat. No. 6,432,940 |
| | 3.beta.,17.beta.-diol | | | | | |
| A39 | N-(substituted glycyl)-2 | | Dipeptidyl | | | U.S. Pat. No. 6,432,969 |
| | cyanopyrrolidines | | peptidase-IV | | | |
| | | | Inhibitor | | | |
| A41 | Protein tyrosine | | | | | U.S. Pat. No. 6,410,586 |
| | phosphatase inhibitor | | | | | |
| A42 | | | Corticotropin | | | U.S. Pat. No. 6,403,599 |
| | | | releasing factor | | | |
| | | | antagonists | | | |
| A43 | Insulin-like growth factor | | | | | U.S. Pat. No. 6,358,925 |
| | I (IGF-I) | | | | | |
| A44 | | | Isoflavones | | | U.S. Pat. No. 6,340,703 |
| A45 | | | Oxytocin and | | | U.S. Pat. No. 6,333,313 |
| | | | Oxytocin | | | |
| | | | analogs | | | |
| A46 | | | Retinoid | | | U.S. Pat. No. 6,326,397 |
| | | | Antagonists | | | |
| A47 | | | Steroids | | | U.S. Pat. No. 6,313,180 |
| A48 | 11.beta.-aryl-substituted | | | | | U.S. Pat. No. 6,229,029 |
| | 14,17-ethanoestratriene | | | | | |
| A49 | B-nor-6-thiaequilenin | | | | | U.S. Pat. No. 6,218,425 |
| A51 | Nitric oxide donor | | | | | U.S. Pat. No. 6,133,320 |
| A52 | Nitric oxide synthase | | | | | U.S. Pat. No. 6,133,320 |
| | substrate | | | | | |
| A53 | (4-arylsulfonylamino)- | | | | | U.S. Pat. No. 6,087,392 |
| | tetrahydropyran-4- | | | | | |
| | carboxilic acid | | | | | |
| | hydroxamide derivative | | | | | |
| A54 | Minimal orthesis | | | | | U.S. Pat. No. 6,063,047 |
| A55 | | | Cytokines | | | U.S. Pat. No. 6,046,033 |
| A56 | 3-hydroxy-4-pyrone | | | | | U.S. Pat. No. 5,998,397 |
| A57 | | | Polynucleotides | | | U.S. Pat. No. 5,935,814 |
| A58 | Epoxysuccinic acid | | | | | U.S. Pat. No. 5,843,992 |
| | derivatives | | | | | |
| A59 | Triaryl-ethylene | | | | | U.S. Pat. No. 5,691,384 |
| | derivatives | | | | | |
| A60 | | | Fibrinogen- | | | U.S. Pat. No. 5,614,535 |
| | | | binding inhibitors | | | |
| A61 | 4-aryloxy-5-hydroxy- | | | | | U.S. Pat. No. 5,466,830 |
| | 2(5H)-furanones | | | | | |
| A62 | 3,4-diarylchromas | | | | | U.S. Pat. No. 5,464,862 |
| A63 | Droloxifene | | Antiresorptive | | | U.S. Pat. No. 6,376,502 |
| | | | Agent | | | |
| A64 | Tamoxifen | | Antiresorptive | | | U.S. Pat. No. 6,376,502 |
| | | | Agent | | | |
| A65 | 4-hydroxy-tamoxifen | | Antiresorptive | | | U.S. Pat. No. 6,376,502 |
| | | | Agent | | | |
| A66 | Toremifene | | Antiresorptive | | | U.S. Pat. No. 6,376,502 |
| | | | Agent | | | |
| A67 | Levormeloxifene | | Antiresorptive | | | U.S. Pat. No. 6,376,502 |
| | | | Agent | | | |
| A68 | Idoxifene | | Antiresorptive | | | U.S. Pat. No. 6,376,502 |
| | | | Agent | | | |
| A69 | 6-(4-hydroxy-phenyl)-5- | | Antiresorptive | | | U.S. Pat. No. 6,376,502 |
| | [4-(2-piperidin-1-yl- | | Agent | | | |
| | ethoxy)-benzyl]- | | | | | |
| | naphthalen-2-ol | | | | | |
| A70 | {4-[2-(2-aza-bicyclo[2.2. | | Antiresorptive | | | U.S. Pat. No. 6,376,502 |
| | 1]hept-2-yl)-ethoxy]- | | Agent | | | |
| | phenyl}-[6-hydroxy-2-(4- | | | | | |
| | hydroxy-phenyl)- | | | | | |
| | benzo[b]thiophen-3-yl]- | | | | | |
| | methanone | | | | | |
| A71 | 3-(4-(1,2-diphenyl-but-1- | | Antiresorptive | | | U.S. Pat. No. 6,376,502 |
| | enyl)-phenyl)-acrylic acid | | Agent | | | |
| A72 | 2-(4-methoxy-phenyl)-3- | | Antiresorptive | | | U.S. Pat. No. 6,376,502 |
| | [4-(2-piperidin-1-yl- | | Agent | | | |
| | ethoxy)-phenoxy]- | | | | | |
| | benzo[b]thiophen-6-ol | | | | | |
| A73 | cis-6-(4-fluoro-phenyl)-5- | | Antiresorptive | | | U.S. Pat. No. 6,376,502 |
| | [4-(2-piperidin-1-yl- | | Agent | | | |
| | ethoxy)-phenyl]-5,6,7,8- | | | | | |
| | tetrahydro-naphthalene- | | | | | |
| | 2-ol | | | | | |
| A74 | (-)-cis-6-phenyl-5-[4-(2- | | Antiresorptive | | | U.S. Pat. No. 6,376,502 |
| | pyrrolidin-1-yl-ethoxy)- | | Agent | | | |
| | phenyl]-5,6,7,8- | | | | | |
| | tetrahydro-naphthalene- | | | | | |
| | 2-ol | | | | | |
| A75 | cis-6-phenyl-5-[4-(2- | | Antiresorptive | | | U.S. Pat. No. 6,376,502 |
| | pyrrolidin-1-yl-ethoxy)- | | Agent | | | |
| | phenyl]-5,6,7,8- | | | | | |
| | tetrahydro-naphthalene- | | | | | |
| | 2-ol | | | | | |
| A76 | cis-1-[6′- | | Antiresorptive | | | U.S. Pat. No. 6,376,502 |
| | pyrrolodinoethoxy-3′- | | Agent | | | |
| | pyridyl]-2-phenyl-6- | | | | | |
| | hydroxy-1,2,3,4- | | | | | |
| | tetrahydronaphthalene | | | | | |
| A77 | 1-(4′- | | Antiresorptive | | | U.S. Pat. No. 6,376,502 |
| | pyrrolidinoethoxyphenyl)- | | Agent | | | |
| | 2-(4″-fluorophenyl)-6- | | | | | |
| | hydroxy-1,2,3,4- | | | | | |
| | tetrahydroisoquinoline | | | | | |
| A78 | cis-6-(4-hydroxyphenyl)- | | Antiresorptive | | | U.S. Pat. No. 6,376,502 |
| | 5-[4-(2-piperidin-1-yl- | | Agent | | | |
| | ethoxy)-phenyl]-5,6,7,8- | | | | | |
| | tetrahydro-naphthalene- | | | | | |
| | 2-ol | | | | | |
| A79 | 1-(4′- | | Antiresorptive | | | U.S. Pat. No. 6,376,502 |
| | pyrrolidinolethoxyphenyl)- | | Agent | | | |
| | 2-phenyl-6-hydroxy- | | | | | |
| | 1,2,3,4-tetrahydroisoquinoline | | | | | |
| A80 | β-alanine derivatives | | | | | U.S. Pat. No. 6,576,637 |
| A81 | Centchroman | | Antiresorptive | | | |
| | | | Agent | | | |
| A82 | Methanebisphosphonic | | Bisphosponate | | | U.S. Pat. No. 6,579,860 |
| | Acid | | | | | |
| A83 | Thienyl substituted | | | | | U.S. Pat. No. 6,566,366 |
| | acylguanidines | | | | | |
| A84 | Myb induced myeloid | | | | | U.S. Pat. No. 6,566,333 |
| | protein-1 | | | | | |
| A85 | | | Bicyclic amino | | | U.S. Pat. No. 6,556,144 |
| | | | acids | | | |
| A86 | Fused thiophene | | Interleukin-6 | | | U.S. Pat. No. 6,555,555 |
| | derivative | | inhibitor | | | |
| A87 | Benzene butyric acid | | | | | U.S. Pat. No. 6,544,969 |
| A88 | | | Phosphodiesterase | | | U.S. Pat. No. 6,531,498 |
| | | | VII inhibitors | | | |
| A89 | Dibenzoazulene | | | | | U.S. Pat. No. 6,521,646 |
| A90 | 3-cyanoquinolines | | | | | U.S. Pat. No. 6,521,618 |
| A91 | 3 cyano-1,6- | | | | | U.S. Pat. No. 6,521,618 |
| | napthyridines | | | | | |
| A92 | 3-cyano-1,7,- | | | | | U.S. Pat. No. 6,521,618 |
| | napthyridines | | | | | |
| A93 | Benzazepinone | | | | | U.S. Pat. No. 6,521,618 |
| | derivatives | | | | | |
| A94 | | | Vitamin D | | | U.S. Pat. No. 6,544,969 |
| A95 | Fluoride | | | | | U.S. Pat. No. 4,904,478 |
| A96 | Norethindrone | | Progestin | | | U.S. Pat. No. 5,646,137 |
| A97 | Medroxyprogesterone | | Progestin | | | U.S. Pat. No. 5,646,137 |
| A98 | | | Xanthine | | | U.S. Pat. No. 5,436,258 |
| | | | oxidase | | | |
| | | | inhibitors | | | |
| A99 | | | integrin blockers | | | |
| A100 | | | alphaᵥbeta₃ | | | |
| | | | antagonists | | | |
| A101 | Lasofoxifene | | Selective | | | |
| | | | estrogen | | | |
| | | | receptor | | | |
| | | | modulator | | | |
| A102 | | | cathespin K | | | |
| | | | inhibitors | | | |
| A103 | | | ATP proton | | | |
| | | | pump inhibitors | | | |
| A104 | Prenylated | | Novel tripeptide | | | US Patent Application |
| | Pyrophospate | | and tetrapeptide | | | No. 20040121941 |
| | Consuming enzyme | | analogs | | | To Burm; et al. |
| | inhibitors | | | | | |
| A105 | | | Novel | | | US Patent Application |
| | | | heterocycles | | | No. 20020103161 To |
| | | | | | | Weigel; et al. |
| A106 | | | Novel purines | | | US Patent Application |
| | | | | | | No. 20020132819 to |
| | | | | | | Metcalf; et al. |
| A107 | | | Triphenylmethane | | | US Patent Application |
| | | | derivatives | | | No. 20020156301 to |
| | | | | | | Kaneko; et al. |
| A108 | | | Dietary | | | US Patent Application |
| | | | supplement with | | | No. 20030059481 to |
| | | | vitamin D, | | | Krumhar; at al. |
| | | | calcium and | | | |
| | | | osteoblast | | | |
| | | | stimulant | | | |
| A109 | | | 1,3-Dihydroxy- | | | US Patent Application |
| | | | 20,20-dialkyl- | | | No. 20030083319 to |
| | | | vitamin D3 | | | Manchard; et al. |
| | | | analogs | | | |
| A110 | | | Novel | | | US Patent Application |
| | | | pyridopyrimidones | | | No. 20030100572 to |
| | | | | | | Metcalf; et al. |
| A111 | | | Novel | | | US Patent Application |
| | | | quinazolines | | | No. 20030100573 to |
| | | | | | | Wang; et al. |
| A112 | | | Novel quinolines | | | US Patent Application |
| | | | | | | No. 20030105065 to |
| | | | | | | Wang; et al. |
| A113 | | | Novel | | | US Patent Application |
| | | | pyridopyrimidines | | | No. 20030105115 to |
| | | | | | | Metcalf; et al. |
| A114 | | | Novel pyrazolo- | | | US Patent Application |
| | | | and pyrrolo- | | | No. 20030114467 to |
| | | | pyrimidines | | | Shakespeare; et al. |
| A115 | | | skeletal anabolic | | | US Patent Application |
| | | | drugs | | | No. 20030171288 to |
| | | | | | | Stewart, Andrew F. |
| A116 | | | Novel quinolines | | | US Patent Application |
| | | | | | | No. 20030114486 to |
| | | | | | | Metcalf; et al. |
| A117 | | | 3-desoxy vitamin | | | US Patent Application |
| | | | D3 analogs | | | No. 20030130241 to |
| | | | | | | Uskokovic, |
| | | | | | | Milan Radoje. |
| A118 | | | Heterocyclic | | Bristol-Myers | U.S. Pat. No. |
| | | | aromatic | | Squibb | 6,660,760 to Robl, et al. |
| | | | compounds | | | |
| | | | useful as growth | | | |
| | | | hormone | | | |
| | | | secretagogues | | | |
| A119 | | | Quinolinones | | Ariad | U.S. Pat. No. |
| | | | | | Pharmaceuticals | 6,713,462 to Metcalf, et |
| | | | | | | al. |
| A120 | | | Quinolines | | Ariad | U.S. Pat. No. |
| | | | | | Pharmaceuticals | 6,706,699 to Wang, et al. |
| A121 | | | Estrogenic | | Endeavor | U.S. Pat. No. |
| | | | compounds | | Pharmaceuticals | 6,660,726 to Hill, et al. |
| A122 | | | Androgen | | Merck | U.S. Pat. No. |
| | | | receptor | | | 6,645,974 to Hutchinson, |
| | | | modulators | | | et al. |
| A123 | | | Conjugated | | Watson | U.S. Pat. No. |
| | | | estrogens | | Pharmaceuticals | 6,630,166 to Ho. |
| A124 | Prenylated pyrophospate | | Novel peptide- | | | U.S. Pat. No. |
| | consuming enzyme | | like FPP- | | | 6,624,147 to Burm, et al. |
| | inhibitors | | analogues | | | |
| A125 | | | 3-desoxy-vitamin | | Syntex | U.S. Pat. No. |
| | | | D3 analog esters | | | 6,559,138 to Uskokovic. |
| A126 | | | 1,3-dihydroxy- | | Syntex | U.S. Pat. No. |
| | | | 20,20-cycloalkyl- | | | 6,492,353 to Manchand, |
| | | | vitamin D3 | | | et al. |
| | | | analogs | | | |
| A127 | | | Prostaglandin | | Merck | U.S. Pat. No. |
| | | | conjugates | | | 6,121,253 to Han, et al. |
| A128 | | | Vitamin D3 | | Syntex | U.S. Pat. No. |
| | | | analogs with bis | | | 6,030,962 to Manchand, |
| | | | C-20 side chains | | | et al. |
| A129 | | | Fluorinated | | Syntex | U.S. Pat. No. |
| | | | vitamin D3 | | | 5,872,113 to Nestor, et |
| | | | analogs | | | al. |
| A130 | | | Methanedi- | | Toray Industries | U.S. Pat. No. |
| | | | phosphonate | | | 5,683,992 to Kawabe, et |
| | | | derivatives | | | al. |

As described above, several bone disorder treatment agents are available for a combination treatment or prevention therapy comprising one or more bone disorder treatment agents and a Cox-2 inhibitor for treating or preventing bone disorders and bone disorder-related complications.

Bone remodeling consists of two distinct stages: bone resorption and bone formation. During resorption, special cells called osteoclasts dissolve bone tissue and create small cavities. During formation, cells known as osteoblasts fill the cavities with new bone tissue. Bone resorption and bone formation are linked so that they occur in close sequence and, ideally, remain balanced. An imbalance in the bone remodeling cycle causes bone loss that eventually leads to osteoporosis and fracture risk.

For purposes of the present invention, bone disorder treatment agents such as bisphosphonates, for example, can be combined with Cox-2 inhibitors as an effective co-therapy method for treating or preventing bone disorders. Likewise, bone disorder treatment agents such as bisphosphonates, for example, can be combined with Cox-2 inhibitors in novel compositions for treating or preventing bone disorders.

In some embodiments, examples of preferred classes of bone disorder treatment agents capable of treating or preventing the symptoms of bone disorders in combination with a Cox-2 inhibitor include, but are not limited to one or more of antiresorptive compounds, parathyroid hormones, fluorides, and mixtures thereof.

As used herein, the term “antiresorptive compounds” means any compound that slows or stops the bone-resorbing portion of the bone remodeling cycle, but does not necessarily affect the bone formation portion of the cycle. As a result, new formation continues at a greater rate than bone resorption, and bone density may increase over time. Other compounds, known as “anabolic compounds,” increase the rate of the bone formation cycle. Examples of anabolic compounds include, but are not limited to, parathyroid hormones and fluorides.

Examples of preferred antiresorptive compounds capable of treating or preventing the symptoms of bone disorders in combination with a Cox-2 inhibitor include, but are not limited to bisphosphonates, calcitonin, estrogens, selective estrogen receptor modulators, vitamins, or a mixtures thereof.

The bone disorder treatment agent can also be chosen from osteoclast-secreted proteins; growth hormone secretagogues; integrin inhibitors (blockers); interleukin-6 inhibitors; interleukin-12 inhibitors; matrix metalloproteinase inhibitors; phosphodiesterase VII inhibitors; glycosylated polyamines; pyridine compounds; cysteine protease inhibitors; prostaglandin agonists; thiol-containing compounds; amino-alcohol derivatives; dipeptidyl peptidase-IV inhibitors; corticotrophin releasing factor antagonists; isoflavones; oxytocin; oxytocin analogs; retinoid antagonists; steroids; cytokines; polynucleotides; fibrinogen-binding inhibitors; antiresorptive agents; bicyclic amino acids; progestins; xanthine oxidase inhibitors; alphaᵥbeta₃ antagonists; selective estrogen receptor modulators; cathespin K inhibitors; ATP proton pump inhibitors; androgen receptor modulators; and mixtures thereof.

In one embodiment, the bone disorder treatment agent is chosen from 4-aminobutanoic acid derivatives; thienyl substituted acylguanidines; genus anethum extract; halogenated triphenylethylene derivatives; condensed 4,5,6,7-tetrahydrobenzo[C]thiophene, arylalkanoylpyridazine; disulfides; beta-amino acid nitrile; androst-5-ene-3.beta.,17.beta.-diol; N-(substituted glycyl)-2 cyanopyrrolidines; protein tyrosine phosphatase inhibitor; insulin-like growth factor I (IGF-I); 11 .beta.-aryl-substituted 14,17-ethanoestratriene; B-nor-6-thiaequilenin; nitric oxide donors; nitric oxide synthase substrate; novel tripeptide and tetrapeptide analogs; novel heterocycles; novel purines; triphenylmethane derivatives; dietary supplement with vitamin D, calcium and osteoblast stimulant; 1,3-dihydroxy-20, 20-dialkyl-vitamin D3 analogs; novel pyridopyrimidones; novel quinazolines; novel quinolines; novel pyridopyrimidines; novel pyrazolo- and pyrrolo-pyrimidines; skeletal anabolic drugs; 3-desoxy vitamin D3 analogs; heterocyclic aromatic compounds useful as growth hormone secretagogues; quinolinones; estrogenic compounds; conjugated estrogens; novel peptide-like FPP-analogues; 3-desoxy-vitamin D3 analog esters; 1,3-dihydroxy-20,20-cycloalkyl-vitamin D3 analogs; prostaglandin conjugates; vitamin D3 analogs with bis C-20 side chains; fluorinated vitamin D3 analogs; methanediphosphonate derivatives; (4-arylsulfonylamino)-tetrahydropyran-4-carboxilic acid hydroxamide derivatives; 3-hydroxy-4-pyrone, epoxysuccinic acid derivatives, triaryl-thylene derivatives; 4-aryloxy-5-hydroxy-2(5H)-furanones; 3,4-diarylchromas; droloxifene; tamoxifen; 4-hydroxy-tamoxifen; toremeifene; levormeloxifene; idoxifene; 6-(4-hydroxy-phenyl)-5-[4-(2-piperidin-1-yl-ethoxy)-benzyl]-naphthalen-2ol, {4-[2-(2-aza-bicyclo[2.2.1]hept-2-yl)-ethoxy]-phenyl}-[6-hydroxy-2-(4-hydroxy-phenyl)-benzo[b]thiophen-3-yl]-methanone; 3-(4-(1,2-diphenyl-but-1-enyl)-phenyl)-acrylic acid; 2-(4-methoxy-phenyl)-3-[4-(2-piperidin-1-yl-ethoxy)-phenoxy]-benzo[b]thiophen-6-ol; cis-6-(4-fluoro-phenyl)-5-[4-(2-piperidin-1-yl-ethoxy)-phenyl]-5,6,7,8-tetrahydro-naphthalene-2-ol; (−)-cis-6-phenyl-5-[4-(2-pyrrolidin-1-yl-ethoxy)-phenyl]-5,6,7,8-tetrahydro-naphthalene-2-ol; cis-6-phenyl-5-[4-(2-pyrrolidin-1-yl-ethoxy)-phenyl]-5,6,7,8-tetrahydro-naphthalene-2-ol; cis-1-[6′-pyrrolodinoethoxy-3′-pyridyl]-2-phenyl-6-hydroxy-1,2,3,4-tetrahydronaphthalene; 1-(4′-pyrrolidinoethoxyphenyl)-2-(4″-fluorophenyl)-6-hydroxy-1,2,3,4-tetrahydroisoquinoline; cis-6-(4-hydroxyphenyl)-5-[4-(2-piperidin-1-yl-ethoxy)-phenyl]-5,6,7,8-tetrahydro-naphthalene-2-ol; 1-(4′-pyrrolidinolethoxyphenyl)-2-phenyl-6-hydroxy-1,2,3,4-tetrahydroisoquinoline; β-alanine derivatives; centchroman; thienyl substituted acylguanidines; myb induced myeloid protein-1; fused thiophene derivates; benzene butyric acid; isoxazole; dibenzoazulene; 3-cyanoquinolines; 3-cyano-1,6-napthyridines; 3 cyano-1,7,-napthyridines; benzazepinone derivatives; fluorides; norethindrone; medroxyprogesterone; and mixtures thereof.

Examples of suitable bisphosphonates include alendronate, mildronate, olpadronate, ibandronate, risedronate, etidronate, zoledronate, minodronate, tiludronate, cimadronate, incadronate, neridronate, pamidronate, clodronate, menodronate, and mixtures thereof.

In one embodiment, the bisphosphonate is chosen from:
4-amino-1-hydroxybutylidene-1,1-bisphosphonic acid,
N,N-dimethyl-3-amino-1-hydroxypropylidene-1,1-bisphosphonic acid,
1-hydroxy-3-(N-methyl-N-pentylamino)propylidene-1,1-bisphosphonic acid,
1-hydroxy-2-(3-pyridyl)ethylidene-1,1-bisphosphonic acid,
1-hydroxyethylidene-1,1-bisphosphonic acid,
1-hydroxy-3-(1-pyrrolidinyl)propylidene-1,1-bisphosphonic acid,
1-hydroxy-2-(1-imidazolyl)etylidene-1,1-bisphosphonic acid,
1-hydroxy-2-(imidazo[1,2-a]pyridin-3-yl)ethylidene-1,1-bisphosphonic acid,
1-(4-chlorophenylthio)methylidene-1,1-bisphosphonic acid,
1-(cycloheptylamino)methylidene-1,1-bisphosphonic acid,
6-amino-1-hydroxyhexylidene-1,1-bisphosphonic acid, methanebisphosphonic acid, and mixtures thereof.

Also encompassed by the present invention are the estrogen bone disorder treatment agents. Treatment with estrogen and estrogen derivatives is commonly referred to as estrogen replacement therapy (“ERT”). In preferred embodiments, the estrogen is chosen from estradiol, esterified estrogen, estropipate, ethinyl estradiol, conjugated estrogen, conjugated estrogen plus medroxyprogesterone acetate, and mixtures thereof.

In preferred embodiments, the selective estrogen receptor modulator is raloxifene hydrochloride.

It is preferred that the vitamin is chosen from calcium, vitamin D, and mixtures thereof. As used herein, “vitamins” include all vitamins and minerals.

In a preferred embodiment, a Cox-2 inhibitor such as, for example, celecoxib, can be administered to a subject alone or in combination with a bone disorder treatment agent.

In other preferred embodiments, any combination of the Cox-2 inhibitors and bone disorder treatment agents that are described above can be used in the novel methods, compositions, pharmaceutical compositions and kits of the present invention.

Therefore, in one embodiment, one or more of an antiresorptive bone disorder treatment agent is combined with at least one Cox-2 inhibitor. In another embodiment, parathyroid hormone is combined with at least one Cox-2 inhibitor. In still another embodiment, one or more fluoride bone disorder treatment agents are combined with at least one Cox-2 inhibitor.

Further encompassed by the present invention are any combinations of one or more of antiresorptive, parathyroid hormone, and fluoride agents, each independently, or any combinations thereof, that are also combined with at least one Cox-2 inhibitor.

A Cox-2 inhibitor such as, for example, celecoxib, can be combined with any of the aforementioned bone disorder treatment agents cited in table 3, including, for example, alendronate.

In certain aspects, the present invention encompasses a method of preventing or treating bone disorders and bone disorder-related complications in a subject comprising administering to the subject a Cox-2 inhibitor, wherein the Cox-2 inhibitor comprises at least one compound that is chosen from celecoxib, parecoxib, deracoxib, valdecoxib, etoricoxib, meloxicam, lumiracoxib, tilmacoxib, cimicoxib, nimesulide, flosulide, darbufelone, RS 57067, T-614, BMS-347070, S-2474, SVT-2016, CT-3, ABT-963, SC-58125, NS-398, L-745337, RWJ-63556, L-784512, CS-502, LAS-34475, LAS-34555, S-33516, SD-8381, PMI-001, 644784, CS-706, PAC-10549, PAC-10649, prodrugs of any of them, and mixtures thereof. In other embodiments, the present invention encompasses a method of preventing or treating bone disorders and bone disorder-related complications in a subject comprising administering to the subject a Cox-2 inhibitor in combination with one or more bone disorder treatment agents, wherein the Cox-2 inhibitor is chosen from celecoxib, parecoxib, deracoxib, valdecoxib, meloxicam, lumiracoxib, etoricoxib, cimicoxib, RS 57067, T-614, BMS-347070, tilmacoxib, S-2474, SVT-2016, CT-3, ABT-963, SC-58125, nimesulide, flosulide, cimicoxib, NS-398, L-745337, RWJ-63556, L-784512, darbufelone, CS-502, LAS-34475, LAS-34555, S-33516, SD-8381, PMI-001, 644784, CS-706, PAC-10549, PAC-10649, prodrugs of any of them, and mixtures thereof.

In another embodiment, the present invention encompasses a method of preventing or treating bone disorders and bone disorder-related complications in a subject comprising administering to the subject a Cox-2 inhibitor in combination with one or more bone disorder treatment agents, wherein the Cox-2 inhibitor is chosen from celecoxib, parecoxib, deracoxib, valdecoxib, etoricoxib, meloxicam, lumiracoxib, cimicoxib, tilmacoxib, cimicoxib, prodrugs of any of them, and mixtures thereof.

In one embodiment, the present invention encompasses a method of preventing or treating bone disorders and bone disorder-related complications in a subject comprising administering to the subject a Cox-2 inhibitor, wherein the Cox-2 inhibitor is other than a tricyclic Cox-2 selective inhibitor. For example, in certain aspects of the present invention, the method of preventing or treating bone disorders and bone disorder-related complications in a subject may comprise administering to the subject a Cox-2 inhibitor, wherein the Cox-2 inhibitor comprises at least one compound chosen from lumiracoxib and a chromene Cox-2 selective inhibitor, and mixtures thereof.

In another embodiment, the present invention encompasses a method of preventing or treating bone disorders and bone disorder-related complications in a subject that is in need of such prevention or treatment comprising administering to the subject a Cox-2 inhibitor in combination with one or more bone disorder treatment agents, wherein the chromene Cox-2 selective inhibitor is chosen from:
(S)-6-chloro-7-(1,1-dimethylethyl)-2-(trifluoromethyl)-2H-1-benzopyran-3-carboxylic acid,
(2S)-6,8-dimethyl-2-(trifluoromethyl)-2H-chromene-3-carboxylic acid,
(2S)-6-chloro-8-methyl-2-(trifluoromethyl)-2H-chromene-3-carboxylic acid,
(2S)-8-ethyl-6-(trifluoromethoxy)-2-(trifluoromethyl)-2H-chromene-3-carboxylic acid,
(S)-6,8-dichloro-2-(trifluoromethyl)-2H-1-benzopyran-3-carboxylic acid, and
(2S)-6-chloro-5,7-dimethyl-2-(trifluoromethyl)-2H-chromene-3-carboxylic acid, and mixtures thereof.

In another embodiment, the present invention encompasses a method of preventing or treating bone disorders and bone disorder-related complications in a subject comprising administering to the subject a Cox-2 inhibitor in combination with one or more bone disorder treatment agents, wherein the bone disorder treatment agent is chosen from bisphosphonates, calcitonin, estrogens, selective estrogen receptor modulators, parathyroid hormone, vitamins, and mixtures thereof.

In another embodiment, the present invention encompasses a method of preventing or treating bone disorders and bone disorder-related complications in a subject comprising administering to the subject a Cox-2 inhibitor in combination with one or more bone disorder treatment agents, wherein the bone disorder treatment agent is chosen from osteoclast-secreted proteins; growth hormone secretagogues; integrin inhibitors (blockers); interleukin-6 inhibitors; interleukin-12 inhibitors; matrix metalloproteinase inhibitors; phosphodiesterase VII inhibitors; glycosylated polyamines; pyridine compounds; cysteine protease inhibitors; prostaglandin agonists; thiol-containing compounds; amino-alcohol derivatives; dipeptidyl peptidase-IV inhibitors; corticotrophin releasing factor antagonists; isoflavones; oxytocin; oxytocin analogs; retinoid antagonists; steroids; cytokines; polynucleotides; fibrinogen-binding inhibitors; antiresorptive agents; bicyclic amino acids; progestins; xanthine oxidase inhibitors; alphaᵥbeta₃ antagonists; selective estrogen receptor modulators; cathespin K inhibitors; ATP proton pump inhibitors; androgen receptor modulators; and mixtures thereof.

In another embodiment, the present invention encompasses a method of preventing or treating bone disorders and bone disorder-related complications in a subject comprising administering to the subject a Cox-2 inhibitor in combination with one or more bone disorder treatment agents, wherein the bone disorder treatment agent is chosen from 4-aminobutanoic acid derivatives; thienyl substituted acylguanidines; genus anethum extract; halogenated triphenylethylene derivatives; condensed 4,5,6,7-tetrahydrobenzo[C]thiophene, arylalkanoylpyridazine; disulfides; beta-amino acid nitrile; androst-5-ene-3.beta.,17.beta.-diol; N-(substituted glycyl)-2 cyanopyrrolidines; protein tyrosine phosphatase inhibitor; insulin-like growth factor I (IGF-I); 11.beta.-aryl-substituted 14,17-ethanoestratriene; B-nor-6-thiaequilenin; nitric oxide donors; nitric oxide synthase substrate; novel tripeptide and tetrapeptide analogs; novel heterocycles; novel purines; triphenylmethane derivatives; dietary supplement with vitamin D, calcium and osteoblast stimulant; 1,3-dihydroxy-20, 20-dialkyl-vitamin D3 analogs; novel pyridopyrimidones; novel quinazolines; novel quinolines; novel pyridopyrimidines; novel pyrazolo- and pyrrolo-pyrimidines; skeletal anabolic drugs; 3-desoxy vitamin D3 analogs; heterocyclic aromatic compounds useful as growth hormone secretagogues; quinolinones; estrogenic compounds; conjugated estrogens; novel peptide-like FPP-analogues; 3-desoxy-vitamin D3 analog esters; 1,3-dihydroxy-20,20-cycloalkyl-vitamin D3 analogs; prostaglandin conjugates; vitamin D3 analogs with bis C-20 side chains; fluorinated vitamin D3 analogs; methanediphosphonate derivatives; (4-arylsulfonylamino)-tetrahydropyran-4-carboxilic acid hydroxamide derivatives; 3-hydroxy-4-pyrone, epoxysuccinic acid derivatives, triaryl-thylene derivatives; 4-aryloxy-5-hydroxy-2(5H)-furanones; 3,4-diarylchromas; droloxifene; tamoxifen; 4-hydroxy-tamoxifen; toremeifene; levormeloxifene; idoxifene; 6-(4-hydroxy-phenyl)-5-[4-(2-piperidin-1-yl-ethoxy)-benzyl]-naphthalen-2ol, {4-[2-(2-aza-bicyclo[2.2.1]hept-2-yl)-ethoxy]-phenyl}-[6-hydroxy-2-(4-hydroxy-phenyl)-benzo[b]thiophen-3-yl]-methanone; 3-(4-(1,2-diphenyl-but-1-enyl)-phenyl)-acrylic acid; 2-(4-methoxy-phenyl)-3-[4-(2-piperidin-1-yl-ethoxy)-phenoxy]-benzo[b]thiophen-6-ol; cis-6-(4-fluoro-phenyl)-5-[4-(2-piperidin-1-yl-ethoxy)-phenyl]-5,6,7,8-tetrahydro-naphthalene-2-ol; (−)-cis-6-phenyl-5-[4-(2-pyrrolidin-1-yl-ethoxy)-phenyl]-5,6,7,8-tetrahydro-naphthalene-2-ol; cis-6-phenyl-5-[4-(2-pyrrolidin-1-yl-ethoxy)-phenyl]-5,6,7,8-tetrahydro-naphthalene-2-ol; cis-1-[6′-pyrrolodinoethoxy-3′-pyridyl]-2-phenyl-6-hydroxy-1,2,3,4-tetrahydronaphthalene; 1-(4′-pyrrolidinoethoxyphenyl)-2-(4″-fluorophenyl)-6-hydroxy-1,2,3,4-tetrahydroisoquinoline; cis-6-(4-hydroxyphenyl)-5-[4-(2-piperidin-1-yl-ethoxy)-phenyl]-5,6,7,8-tetrahydro-naphthalene-2-ol; 1-(4′-pyrrolidinolethoxyphenyl)-2-phenyl-6-hydroxy-1,2,3,4-tetrahydroisoquinoline; β-alanine derivatives; centchroman; thienyl substituted acylguanidines; myb induced myeloid protein-1; fused thiophene derivates; benzene butyric acid; isoxazole; dibenzoazulene; 3-cyanoquinolines; 3-cyano-1,6-napthyridines; 3 cyano-1,7,-napthyridines; benzazepinone derivatives; fluorides; norethindrone; medroxyprogesterone; and mixtures thereof.

In another embodiment, the present invention encompasses a method of preventing or treating bone disorders and bone disorder-related complications in a subject comprising administering to the subject a Cox-2 inhibitor in combination with one or more bone disorder treatment agents, wherein the bone disorder treatment agent comprises a bisphosphonate, wherein the bisphosphonate is chosen from alendronate, mildronate, olpadronate, ibandronate, risedronate, etidronate, zoledronate, minodronate, tiludronate, cimadronate, incadronate, neridronate, pamidronate, clodronate, menodronate, and mixtures thereof.

In another embodiment, the present invention encompasses a method of preventing or treating bone disorders and bone disorder-related complications in a subject comprising administering to the subject a Cox-2 inhibitor in combination with one or more bone disorder treatment agents, wherein the bone disorder treatment agent comprise a bisphosphonate, wherein the bisphosphonate is chosen from:
4-amino-1-hydroxybutylidene-1,1-bisphosphonic acid,
N,N-dimethyl-3-amino-1-hydroxypropylidene-1,1-bisphosphonic acid,
1-hydroxy-3-(N-methyl-N-pentylamino)propylidene-1,1-bisphosphonic acid,
1-hydroxy-2-(3-pyridyl)ethylidene-1,1-bisphosphonic acid,
1-hydroxyethylidene-1,1-bisphosphonic acid,
1-hydroxy-3-(1-pyrrolidinyl)propylidene-1,1-bisphosphonic acid,
1-hydroxy-2-(1-imidazolyl)etylidene-1,1-bisphosphonic acid,
1-hydroxy-2-(imidazo[1,2-a]pyridin-3-yl)ethylidene-1,1-bisphosphonic acid,
1-(4-chlorophenylthio)methylidene-1,1-bisphosphonic acid,
1-(cycloheptylamino)methylidene-1,1-bisphosphonic acid,
6-amino-1-hydroxyhexylidene-1,1-bisphosphonic acid, methanebisphosphonic acid, and mixtures thereof.

In another embodiment, the present invention encompasses a method of preventing or treating bone disorders and bone disorder-related complications in a subject comprising administering to the subject a Cox-2 inhibitor in combination with one or more bone disorder treatment agents, wherein the bone disorder treatment agent comprises an estrogen, wherein the estrogen is chosen from estradiol, esterified estrogen, estropipate, ethinyl estradiol, conjugated estrogen, conjugated estrogen plus medroxyprogesterone acetate, and mixtures thereof.

In another embodiment, the present invention encompasses a novel therapeutic composition comprising at least one Cox-2 inhibitor and one or more bone disorder treatment agents. In yet another embodiment, the present invention encompasses a pharmaceutical composition comprising a Cox-2 inhibitor, a bone disorder treatment agent, and a pharmaceutically acceptable excipient.

In accordance with the present invention, any composition comprising a Cox-2 inhibitor alone or in combination with a bone disorder treatment agent may be administered to a subject according to standard routes of drug delivery that are well known to one of ordinary skill in the art.

Each of the Cox-2 inhibitors and bone disorder treatment agents of the present invention can be supplied in the form of a salt, or prodrug, if desirable. The Cox-2 inhibitors and bone disorder treatment agents can also be supplied in the form of an isomer, a racemic mixture, or in any other chemical form or combination that, under physiological conditions, still provides for inhibition of the Cox-2 enzyme.

As used herein, the term “prodrug” refers to drug precursor compounds which, following administration to a subject and subsequent absorption, are converted to an active species in vivo via some process, such as a metabolic process. A nonlimiting example of a “prodrug” that will be useful in the methods, combinations and compositions of the present invention is the Cox-2 inhibitor, parecoxib (N-[[4-(5-methyl-3-phenyl-4-isoxazolyl)phenyl]sulfonyl]propanamide).

The Cox-2 inhibitors and bone disorder treatment agents that are useful in the present invention can be of any purity or grade, as long as the preparation is of a quality suitable for pharmaceutical use. The Cox-2 inhibitors and bone disorder treatment agents can be provided in pure form, or it can be accompanied with impurities or commonly associated compounds that do not affect its physiological activity or safety.

The term “pharmaceutically acceptable” is used adjectivally herein to mean that the modified noun is appropriate for use in a pharmaceutical product.

The compounds of the present invention can also be supplied in the form of a pharmaceutically acceptable salt. The terms “pharmaceutically acceptable salt” refer to salts prepared from pharmaceutically acceptable inorganic and organic acids and bases.

Pharmaceutically acceptable inorganic bases include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary salts include aluminum, calcium, lithium, magnesium, potassium, sodium and zinc. Particularly preferred are the ammonium, calcium, magnesium, potassium, and sodium salts.

Salts derived from pharmaceutically acceptable organic non-toxic bases include salts of primary, secondary, and tertiary amines, including in part, trimethylamine, diethylamine, N, N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Illustrative pharmaceutically acceptable acid addition salts of the compounds of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, β-hydroxybutyric, galactaric and galacturonic acids. Preferred pharmaceutically acceptable salts include the salts of hydrochloric acid and trifluoroacetic acid.

All of the above salts can be prepared by those skilled in the art by conventional means from the corresponding compound of the present invention. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p.1418, the disclosure of which is hereby incorporated by reference only with regards to the lists of suitable salts.

The combination of a Cox-2 inhibitor and a bone disorder treatment agent can be provided in a pharmaceutically acceptable carrier or excipient to form a pharmaceutical composition.

Pharmaceutically acceptable carriers and excipients include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The pharmaceutically acceptable carrier can also be selected on the basis of the desired route of administration of the compound. For example, in a preferred embodiment the carrier is suitable for oral administration. In another embodiment, the composition includes a carrier or additional agent that is suitable for promoting delivery of the compound to the brain. Examples of such carriers include those disclosed in U.S. Pat. Nos. 5,604,198; 5,827,819; 5,919,815; 5,955,459 and 5,977,174.

In the present invention, a Cox-2 inhibitor in combination with a bone disorder treatment agent are administered to a subject according to standard routes of drug delivery that are well known to one of ordinary skill in the art. The particular route and dosage of the compounds described herein can depend upon the needs of the particular subject being treated, the type of treatment or prevention, the efficacy of the compound and the degree of disease severity in the subject.

The pharmaceutical compositions may be administered enterally and parenterally. Oral (intra-gastric) is a preferred route of administration. Pharmaceutically acceptable carriers can be in solid dosage forms for the methods of the present invention, which include tablets, capsules, pills, and granules, which can be prepared with coatings and shells, such as enteric coatings and others well known in the art. Liquid dosage forms for oral administration include pharmaceutically acceptable emulsions, solutions, suspensions, syrups, and elixirs.

Enteral administration includes solution, tablets, sustained release capsules, enteric-coated capsules, and syrups. When administered, the pharmaceutical composition may be at or near body temperature.

Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in a mixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the compounds described herein may be formulated with sweetening agents, for example glycerol, sorbitol, or sucrose. Such formulations may also contain a demulcent, a preservative and flavoring and coloring agents.

The subject method of prescribing a Cox-2 inhibitor in combination with a bone disorder treatment agent and compositions comprising the same can also be administered parenterally, either subcutaneously, or intravenously, or intramuscularly, or intrasternally, or by infusion techniques, in the form of sterile injectable aqueous or olagenous suspensions. Parenteral administration includes subcutaneous, intramuscular, intradermal, intramammary, intravenous, and other administrative methods known in the art.

Such suspensions may be formulated according to the known art using those suitable dispersing of wetting agents and suspending agents, which have been mentioned above or other acceptable agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Administration of the compounds described herein can also be by inhalation, in the form of aerosols or solutions for nebulizers. Therefore, in one embodiment, the Cox-2 inhibitor in combination with a bone disorder treatment agent is administered by direct inhalation into the respiratory system of a subject for delivery as a mist or other aerosol or dry powder. Delivery of drugs or other active ingredients directly to the subject's lungs provides numerous advantages including, providing an extensive surface area for drug absorption, direct delivery of therapeutic agents to the disease site in the case of regional drug therapy, eliminating the possibility of drug degradation in the subject's intestinal tract (a risk associated with oral administration), and eliminating the need for repeated subcutaneous injections.

Administration of the compositions of the present invention can also be rectally, in the form of suppositories prepared by mixing the drug with a suitable non-irritating excipient which is solid at ordinary temperature, but liquid at the rectal temperature and will therefore, melt in the rectum to release the drug. Such materials are cocoa butter and polyethylene glycols.

Administration may also be by transvaginal delivery through the use of an intravaginal device. Transvaginal delivery may be desirable for many certain subjects because 10 to 30 times more treatment agent can be delivered transvaginally as can be delivered orally due to the absorption from the vagina, which far exceeds the absorption of drugs from the gastrointestinal tract. Further, vaginal administration generally avoids major problems connected with oral administration, such as gastric and esophageal reflux and ulceration.

Also encompassed by the present invention is buccal or “sub-lingual” administration, which includes lozenges or a chewable gum comprising the compounds, set forth herein. The compounds can be deposited in a flavored base, usually sucrose, and acacia or tragacanth, and pastilles comprising the compounds in an inert base such as gelatin and glycerin or sucrose and acacia.

The prevent invention further encompasses intranasal administration comprising the compounds set forth herein. Intranasal dosage forms include, but are not limited to, aerosols, drops, gels, powders, and mixtures thereof.

Other methods for administration of the compounds described herein include dermal patches that release the medicaments directly into a subject's skin.

Topical delivery systems are also encompassed by the present invention and include ointments, powders, sprays, creams, jellies, collyriums, solutions or suspensions.

The compositions of the present invention can optionally be supplemented with additional agents such as, for example, viscosity enhancers, preservatives, surfactants and penetration enhancers. Viscosity-building agents include, for example, polyvinyl alcohol, polyvinyl pyrrolidone, methylcellulose, hydroxy propyl methylcellulose, hydroxyethyl cellulose, carboxymethyl cellulose, hydroxy propyl cellulose or other agents know to those skilled in the art. Such agents are typically employed at a level of from 0.01% to 2% by weight.

Preservatives can be optionally employed to prevent microbial contamination of the compositions described herein. Suitable preservatives include, but are not limited to, polyquaternium-1, benzalkonium chloride, thimerosal, chlorobutanol, methyl paraben, propyl paraben, phenylethyl alcohol, edetate disodium, sorbic acid, or other agents known to those skilled in the art. The use of polyquaternium-1 as the antimicrobial preservative is preferred. Typically, such preservatives are employed at a level of from 0.001% to 1.0% by weight.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g., Pluronic F-68, F-84 and P-103), cyclodextrin, or other agents known to those skilled in the art. Typically, such co-solvents are employed at a level of from 0.01% to 2% by weight.

A penetration enhancer is an agent used to increase the permeability of the skin to an active agent to increase the rate at which the drug diffuses through the skin and enters the tissues and bloodstream. Examples of penetration enhancers suitable for use with the compositions of the present invention include, but are not limited to, alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and mixtures thereof.

Pharmaceutically acceptable excipients and carriers encompass all the foregoing and the like. The above considerations concerning effective formulations and administration procedures are well known in the art and are described in standard textbooks. See e.g. Gennaro, A. R., Remington: The Science and Practice of Pharmacy, 20^{th} Edition, (Lippincott, Williams and Wilkins), 2000;Hoover, John E., Remington's Pharmaceutical Sciences, Mack Publishing Co., Easton Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

For purposes of the present invention, it is preferred that the amount of a Cox-2 inhibitor that is administered to a subject comprises an effective amount of a Cox-2 inhibitor. It is further preferred that the amount of a Cox-2 inhibitor and the amount of a bone disorder treatment agent together comprise an effective amount of the combination of the two treatment agents. Still further preferred is that the amount of the monotherapy with the Cox-2 inhibitor comprise a therapeutically amount of the Cox-2 inhibitor. Further preferred is that the amount of the co-therapy with the Cox-2 inhibitor and bone disorder treatment agent comprises a therapeutically effective amount of the co-therapy.

Thus, the present invention encompasses a method of preventing and treating a bone disorder and a bone disorder-related complication in a subject in need of such prevention or treatment, the method comprising administering an amount of a Cox-2 inhibitor and an amount of a bone disorder treatment agent wherein the amount of the Cox-2 inhibitor and the amount of the bone disorder treatment agent together comprise a therapeutically effective amount.

As used herein, an “effective amount” means the dose or amount to be administered to a subject and the frequency of administration to the subject, which is readily determined by one having ordinary skill in the art, by the use of known techniques and by observing results obtained under analogous circumstances.

In determining the effective amount or dose, a number of factors are considered by the attending diagnostician, including, but not limited to, the potency and duration of action of the compounds used, the nature and severity of the illness to be treated, as well as the sex, age, weight, general health and individual responsiveness of the patient to be treated, and other relevant circumstances.

As used herein, the terms “therapeutically effective” are intended to qualify the amount of an agent for use in therapy that will achieve the goal of preventing, or improvement in the severity of, the disorder being treated, while avoiding adverse side effects typically associated with alternative therapies. A bone disorder symptom or a bone disorder-related complication symptom is considered ameliorated or improved if any benefit is achieved, no matter how slight.

For example, any reduction in the rate of bone loss, increase in the rate of bone formation, increase in bone mineral density or an increase in the strength of the bone in a patient suffering from a bone disorder such as osteoporosis would be considered an ameliorated symptom. Likewise, any improvement in the skeletal posture or reduction of skeletal abnormalities that result from a bone disorder would also be considered amelioration of a bone disorder. Furthermore, any reduction in symptom severity of a bone disorder-related complication is considered an ameliorated symptom.

As used herein, the terms “prophylactically effective” refer to an amount of a Cox-2 inhibitor alone or in combination with a treatment agent that causes a decrease in the frequency of incidence of bone disorders or a bone disorder-related complication. The term “prophylactic” refers to the prevention of bone disorders or a bone disorder-related complication, whereas the term “therapeutic” refers to the effective treatment of an existing disorder such as bone disorders or a bone disorder-related complication.

It will be appreciated that the amount of the Cox-2 inhibitor and the bone disorder treatment agent required for use in the treatment or prevention of bone disorders and bone disorder-related complications will vary within wide limits and will be adjusted to the individual requirements in each particular case. In general, for administration to adults, an appropriate daily dosage is described herein, although the limits that are identified as being preferred may be exceeded if expedient. The daily dosage can be administered as a single dosage or in divided dosages.

The appropriate dosage level of a Cox-2 inhibitor will generally be from about 0.01 mg per kg to about 140 mg per kg subject body weight per day, which may be administered in single or multiple doses. Preferably, the dosage level will be about 0.1 mg/kg to about 25 mg/kg per day; more preferably about 0.5 mg/kg to about 10 mg/kg per day.

In larger mammals, for example humans, a typical indicated dose is about 0.5 mg to 7 grams orally per day. A compound may be administered on a regimen of several times per day, for example 1 to 4 times per day, preferably once or twice per day.

The amount of the Cox-2 inhibitor that may be combined with the carrier materials to produce a single dosage form will vary depending upon the host treated and the particular mode of administration. For example, a formulation intended for the oral administration of humans may contain from 0.5 mg to 7 g of active agent compounded optionally with an appropriate and convenient amount of carrier material, which may vary from about 5 to about 95 percent of the total composition. Dosage unit forms for the Cox-2 inhibitor will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

The dosage level of a bone disorder treatment agent will necessarily depend on the particular agent that is used. However, in general, the appropriate dosage level of a bone disorder treatment agent will generally be from about 0.0001 mg per kg to about 1000 mg per kg subject body weight per day, which may be administered in single or multiple doses. Preferably, the dosage level will be about 0.001 mg per kg to about 100 mg per kg per day; more preferably about 0.01 mg per kg to about 50 mg per kg per day; even more preferably about 0.1 mg per kg to about 10 mg per kg subject body weight.

A combination therapy comprising a bone disorder treatment agent that is intended for the oral administration of humans may contain from about 10 micrograms to about 10 grams of the bone disorder treatment agent, optionally compounded with an appropriate and convenient amount of carrier material, which may vary from about 5 to about 95 percent of the total composition. More preferably, the bone disorder treatment agent is dosed at between about 0.01 mg and about 1 gram. Even more preferably, the bone disorder treatment agent is dosed at between about 0.1 mg and about 500 mg. Even more preferably still, the bone disorder treatment agent is dosed at between about 1 mg and about 100 mg.

The exact dosage and regimen for administering a Cox-2 inhibitor alone or in combination with a bone disorder treatment agent will necessarily depend upon the potency and duration of action of the compounds used, the nature and severity of the illness to be treated, as well as the sex, age, weight, general health and individual responsiveness of the patient to be treated, and other relevant circumstances. Those skilled in the art will appreciate that dosages may also be determined with guidance from Goodman & Gilman's The Pharmacological Basis of Therapeutics, Ninth Edition (1996), Appendix II, pp.1707-1711.

The effectiveness of a particular dosage of a Cox-2 inhibitor alone or in combination with a bone disorder treatment agent can be determined by monitoring the effect of a given dosage on the progress or prevention of a particular bone disorder.

For example, one method to detect the extent to which a subject is suffering from a bone disorder, such as osteoporosis, is to test the bone mineral density (“BMD”). There are several different methods for testing bone mineral density, all of which are painless, noninvasive, safe, and becoming more readily available. Different BMD tests are available for different areas of the body, including hips, spine, wrist, finger, shin bone, or heel. Dual Energy X-ray Absorptiometry (DXA) is one method available to measure the BMD of a patient's spine, hip, or total body. Quantitative Computed Tomography (QCT) is another available testing apparatus and it also determines the BMD at the spine.

The results of the BMD tests are compared to two different standards. The first is the “young normal,” or T-score, which compares the patient's BMD to the optimal or peak density of a 30-year old healthy adult in order to determine the patient's fracture risk, which increases as BMD falls below young-normal levels. The second standard is the “aged-matched,” or Z-score, which compares the patient's BMD to what is expected in someone of the same age and body size. Among older adults, however, low BMD is common, so comparison with age-matched norms can be misleading.

The difference between a patient's BMD and that of a healthy young adult is referred to as a standard deviation (SD). As outlined in the World Health Organization's diagnostic categories, individuals whose T-score is within one standard deviation of the “norm” are considered to have normal bone density. Scores below the “norm” are indicated by negative numbers. The World Health Organization (“WHO”) defines osteoporosis based on the following results in Table 4:

TABLE 4
WHO Osteoporosis Definitions Based on BMD Test Results

| | Results of BMD Test Compared to |
|---|---|
| Classification | “Young Normal” |
| Normal | Between −1 and +1 SD |
| Low Bone Mass (Osteopenia) | Between −1 and −2.5 SD |
| Osteoporosis | More than −2.5 SD |
| Severe (Established) Osteoporosis | More and −2.5 SD and there has |
| | been more than one osteoporotic |
| | fracture. |

For most BMD tests, −1 SD equals a 10-12 percent decrease in bone density.

As used herein, the term “subject” for purposes of treatment includes any subject, and preferably is a subject who is in need of the treatment of bone disorders, or who needs treatment of a bone disorder-related complication. For purposes of prevention, the subject is any subject, and preferably is a subject that is at risk for, or is predisposed to, developing a bone disorder or a bone disorder-related complication. The subject is typically an animal, and yet more typically is a mammal. “Mammal”, as that term is used herein, refers to any animal classified as a mammal, including humans, domestic and farm animals, zoo, sports, or pet animals, such as dogs, horses, cats, cattle, etc. Preferably, the mammal is a human.

As used herein, the terms “subject is one that is in need of the prevention or treatment of a bone disorder and/or bone disorder-related complication” refer to any subject who is suffering from or is predisposed to bone disorders or any bone disorder-related complication described herein. The terms “subject is one that is in need of the prevention or treatment of a bone disorder and/or bone disorder-related complication” also refer to any subject that requires a lower dose of conventional bone disorder treatment agents. In addition, the terms “subject is one that is in need of the prevention or treatment of a bone disorder and/or bone disorder-related complication” means any subject who requires a reduction in the side-effects of a bone disorder treatment agent. Furthermore, the terms “subject is one that is in need of the prevention or treatment of a bone disorder and/or bone disorder-related complication” means any subject who requires improved tolerability to any bone disorder treatment agent for bone disorders therapy.

In other preferred embodiments, the present invention encompasses a kit for preventing or treating bone disorders or a bone disorder-related complication in a subject comprising one dosage form comprising a Cox-2 inhibitor and a second dosage form comprising a bone disorder treatment agent.

A therapy comprising a Cox-2 inhibitor alone and in combination with a bone disorder treatment agent encompasses the treatment and prevention of such bone disorder symptoms as, for example, low BMD, bone fracture, skeletal distortion, and pain and inflammation of the bone in a subject suffering from such symptoms.

As used herein, the terms “bone disorder” is defined as having any disorder or disease of the bone or even a post-surgical condition of the bone. Bone disorders include any condition of the bone that is not normal in a healthy 30 year old adult, since it is at this point that humans generally reach their peak, or optimal, bone mineral density. As used herein, the term “bone” includes any component or structure found within or on the skeletal structure including, but not limited to, bone tissue, cartilage, bone marrow, teeth, nails, and other hard tissue within the body.

The terms “bone disorder” also include any complications that arise from having such a disorder. For example, bone fracture may develop from a prolonged untreated bone disorder. Thus, the terms “bone disorder,” “bone disorder complication” and “bone disorder-related complication,” used interchangeably herein, includes any subsequent disease, disorder, injury or condition that may arise from having a bone disorder. The term “bone disorder-related complication” refers to any condition where developing a bone disorder is a risk factor for developing health complications.

For example, a bone fracture may arise from having a bone disorder, or it may arise from a trauma. If a bone disorder, such as osteoporosis, is left untreated, the deterioration of the bone can, over time, result in bone fractures, which can lead to a bone disorder-related complication, such as pain, inflammation, and decreased mobility. However, the compositions and methods of the present invention may prevent or treat such a complication by reducing both the original bone loss and the pain and inflammation. A Cox-2 inhibitor alone or in combination with a bisphosphonate or an estrogen agent would be an example of a novel composition and method suitable for treating the bone disorder-related complication of bone fracture and thus, eventual pain and inflammation.

Bone disorders may arise in a subject via several determinants including decreased hormone production, inadequate consumption of vitamins, infectious agents, causative agents and genetics. The methods and compositions of the present invention are intended to treat a subject suffering from a bone disorder regardless of how the disorder first arose.

In one embodiment, bone disorders that may be treated with the compositions and methods described herein, include one or more of, but are not limited to, osteopenia, osteoporosis, Paget's disease (osteitis deformans), bone degradation, bone weakening, skeletal distortion, low bone mineral density, scoliosis, osteomalacia, osteomyelitis, osteogenesis imperfecta, osteopetrosis, enchondromatosis, osteochondromatosis, achondroplasia, avascular necrosis, fibrous dysplasia, periodontal disease, hyperparathyroidism (osteitis fibrosa cystica), hypophosphatasia, fibrodysplasia ossificans progressive, bone fractures, bone breaks, and pain and inflammation of the bone.

Preferably, the methods and compositions of the present invention encompass the prevention or treatment of the bone disorder, osteoporosis.

In another embodiment, several bone disorder-related complications may also be treated or prevented according to the methods and compositions described herein. Having a bone disorder predisposes a subject to certain health risks that increase as the severity of a subject's bone disorder increases.

Increased health complications incident to having a bone disorder include bone disorder-related complications such as, but are not limited to, bone fracture, pain, inflammation, and including any other disorders or complications that are amenable to amelioration through inhibition of the Cox-2 enzyme alone or in combination with administration to a subject in need of such treatment of a bone disorder treatment agent referred to herein.

The methods and compositions of the present invention not only encompass the prevention or treatment of bone disorders and bone disorder-related disorders in humans, but also in several animals. For example, many animals also suffer adverse consequences related to bone disorders. Moreover, many bone disorders in dogs respond to the same treatment used in humans. Accordingly, besides being useful for humans, the methods and compositions of the present invention also encompass the treatment and prevention of bone disorders and bone disorder-related disorders in other mammals, including horses, dogs, cats, rats, mice, sheep, pigs, cattle, hamsters, gerbils, and the like.

The following examples describe embodiments of the invention. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples. In the examples, all percentages are given on a weight basis unless otherwise indicated.

### EXAMPLE 1

This example shows the preparation of celecoxib.

Step 1: Preparation of 1-(4-methylphenyl)-4,4,4-trifluorobutane-1,3-dione.

Following the disclosure provided in U.S. Pat. No. 5,760,068, 4′-Methylacetophenone (5.26 g, 39.2 mmol) was dissolved in 25 mL of methanol under argon and 12 mL (52.5 mmol) sodium methoxide in methanol (25%) was added. The mixture was stirred for 5 minutes and 5.5 mL (46.2 mmol) ethyl trifluoroacetate was added. After refluxing for 24 hours, the mixture was cooled to room temperature and concentrated. 100 mL 10% HCl was added and the mixture extracted with 4×75 mL ethyl acetate. The extracts were dried over MgSO₄, filtered and concentrated to afford 8.47 g (94%) of a brown oil which was carried on without further purification.

Step 2: Preparation of 4-[5-(4-methylphenyl)-3-(trifluoromethyl)-1H-pyrazol-1-yl]benzenesulfonamide.

To the dione from Step 1 (4.14 g, 18.0 mmol) in 75 mL absolute ethanol, 4.26 g (19.0 mmol) 4-sulphonamidophenylhydrazine hydrochloride was added. The reaction was refluxed under argon for 24 hours. After cooling to room temperature and filtering, the reaction mixture was concentrated to afford 6.13 g of an orange solid. The solid was recrystallized from methylene chloride/hexane to give 3.11 g (8.2 mmol, 46%) of the product as a pale yellow solid, having a melting point (mp) of 157°-159° C.; and a calculated composition of C₁₇H₁₄N₃O₂SF₃; C, 53.54; H, 3.70; N, 11.02. The composition that was found by analysis was: C, 53.17;H, 3.81; N, 10.90.

### EXAMPLE 2

This example illustrates the production of a composition containing celecoxib and a selective estrogen receptor modulator, such as Raloxifene, and of a pharmaceutical composition containing the combination.

Raloxifene is available in the form of tablets under the trade name Evista® from Eli Lilly and Company, Indianapiolis, Ind. Celecoxib can be prepared as described in Example 1, or it can be obtained under the trade name CELEBREX® from Pharmacia Corporation, Peapack, N.J.

A therapeutic composition of the present invention can be formed by intermixing raloxifene hydrochloride (60 g, available as Evista®, from Eli Lilly and Company), and 4-[5-(4-methylphenyl)-3-(trifluoromethyl)-1H-pyrazol-1-yl]benzenesulfonamide (200 g, as produced in Comparative Example 1, or as available from Pharmacia Corporation, Peapack, N.J., under the tradename CELEBREX®), in a laboratory mill or mixing device suitable for intimate mixing of powders without substantial generation of shear or temperature sufficient to degrade either of the two compounds. After mixing, the combination of raloxifene and celecoxib forms a therapeutic composition that is sufficient for the production of about 1000 human single dose units. Each single dose unit contains about 60 mg of raloxifene and about 200 mg of celecoxib.

If desirable, a solid carrier and other materials may be intermixed with the therapeutic composition to form a pharmaceutical composition and the resulting pharmaceutical composition may be formed into capsules for human consumption, for example, by conventional capsule-forming equipment, where each capsule contains 60 mg of raloxifene and 200 mg celecoxib.

Therapeutic and pharmaceutical compositions comprising a combination of any of the Cox-2 selective inhibitors and any bone disorder treatment agent active ingredients that are described above can be formed by similar methods.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense. In addition, it should be understood that aspects of the various embodiments may be interchanged both in whole or in part.

1. A method of preventing or treating bone disorders and bone disorder-related complications in a subject comprising administering to the subject a Cox-2 inhibitor, wherein the Cox-2 inhibitor comprises at least one compound that is chosen from celecoxib, parecoxib, deracoxib, valdecoxib, etoricoxib, meloxicam, lumiracoxib, tilmacoxib, cimicoxib, nimesulide, flosulide, darbufelone, RS 57067, T-614, BMS-347070, S-2474, SVT-2016, CT-3, ABT-963, SC-58125, NS-398, L-745337, RWJ-63556, L-784512, CS-502, LAS-34475, LAS-34555, S-33516, SD-8381, PMI-001, 644784, CS-706, PAC-10549, PAC-10649, prodrugs of any of them, and mixtures thereof.

2. A method of preventing or treating bone disorders and bone disorder-related complications in a subject comprising administering to the subject a Cox-2 inhibitor in combination with one or more bone disorder treatment agents.

3. The method according to claim 2, wherein the subject is one that is in need of the prevention or treatment of a bone disorder and/or bone disorder-related complication.

4. The method according to claim 2, wherein the Cox-2 inhibitor comprises a non-steroidal anti-inflammatory drug.

5. The method according to claim 2, wherein the Cox-2 inhibitor comprises at least one compound that is chosen from ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, nitroflurbiprofen, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam and 2-fluoro-a-methyl[1,1′-biphenyl]-4-acetic acid, 4-(nitrooxy)butyl ester, and mixtures thereof.

6. The method according to claim 2, wherein the Cox-2 inhibitor comprises a Cox-2 selective inhibitor.

7. The method according to claim 6, wherein the Cox-2 selective inhibitor comprises at least one compound that is chosen from celecoxib, parecoxib, deracoxib, valdecoxib, etoricoxib, meloxicam, rofecoxib, lumiracoxib, tilmacoxib, cimicoxib, nimesulide, flosulide, darbufelone, RS 57067, T-614, BMS-347070, S-2474, SVT-2016, CT-3, ABT-963, SC-58125, NS-398, L-745337, RWJ-63556, L-784512, CS-502, LAS-34475, LAS-34555, S-33516, SD-8381, PMI-001, 644784, CS-706, PAC-10549, PAC-10649, prodrugs of any of them, and mixtures thereof.

8. The method according to claim 6, wherein the Cox-2 selective inhibitor comprises at least one compound that is chosen from celecoxib, parecoxib, deracoxib, valdecoxib, etoricoxib, meloxicam, lumiracoxib, tilmacoxib, cimicoxib, nimesulide, flosulide, darbufelone, RS 57067, T-614, BMS-347070, S-2474, SVT-2016, CT-3, ABT-963, SC-58125, NS-398, L-745337, RWJ-63556, L-784512, CS-502, LAS-34475, LAS-34555, S-33516, SD-8381, PMI-001, 644784, CS-706, PAC-10549, PAC-10649, prodrugs of any of them, and mixtures thereof.

9. The method according to claim 6, wherein the Cox-2 selective inhibitor comprises a tricyclic Cox-2 selective inhibitor.

10. The method according to claim 9, wherein the tricyclic Cox-2 selective inhibitor comprises at least one compound that is chosen from celecoxib, parecoxib, deracoxib, valdecoxib, etoricoxib, tilmacoxib, cimicoxib, prodrugs of any of them, and mixtures thereof.

11. The method according to claim 6, wherein the Cox-2 selective inhibitor comprises at least one compound that is other than a tricyclic Cox-2 selective inhibitor.

12. The method according to claim 11, wherein the Cox-2 selective inhibitor comprises at least one compound chosen from a chromene Cox-2 selective inhibitor, lumiracoxib, RS 57067, NS-398, BMS 347070, ABT-963, SD-8381, PAC-10549, PAC-10649, prodrugs of any of them, and mixtures thereof.

13. The method according to claim 11, wherein the chromene Cox-2 selective inhibitor comprises at least one compound chosen from 6-chloro-2-trifluoromethyl-2H-1-benzopyran-3-carboxylic acid, 6-chloro-7-methyl-2-trifluoromethyl-2H-1-benzopyran-3-carboxylic acid, 8-(1-methylethyl)-2-trifluoromethyl-2H-1-benzopyran-3-carboxylic acid, 6-chloro-7-(1,1-dimethylethyl)-2-trifluoromethyl-2H-1-benzopyran-3-carboxylic acid, 6-chloro-8-(1-methylethyl)-2-trifluoromethyl-2H-1-benzopyran-3-carboxylic acid, 2-trifluoromethyl-3H-naphthopyran-3-carboxylic acid, 7-(1,1-dimethylethyl)-2-trifluoromethyl-2H-1-benzopyran-3-carboxylic acid, 6-bromo-2-trifluoromethyl-2H-1-benzopyran-3-carboxylic acid, 8-chloro-2-trifluoromethyl-2H-1-benzopyran-3-carboxylic acid, 6-trifluoromethoxy-2-trifluoromethyl-2H-1-benzopyran-3-carboxylic acid, 5,7-dichloro-2-trifluoromethyl-2H-1-benzopyran-3-carboxylic acid, 8-phenyl-2-trifluoromethyl-2H-1-benzopyran-3-carboxylic acid, 7,8-dimethyl-2-trifluoromethyl-2H-1-benzopyran-3-carboxylic acid, 6,8-bis(dimethylethyl)-2-trifluoromethyl-2H-1-benzopyran-3-carboxylic acid, 7-(1-methylethyl)-2-trifluoromethyl-2H-1-benzopyran-3-carboxylic acid, 7-phenyl-2-trifluoromethyl-2H-1-benzopyran-3-carboxylic acid, 6-chloro-7-ethyl-2-trifluoromethyl-2H-1-benzopyran-3-carboxylic acid, 6-chloro-8-ethyl-2-trifluoromethyl-2H-1-benzopyran-3-carboxylic acid, 6-chloro-7-phenyl-2-trifluoromethyl-2H-1-benzopyran-3-carboxylic acid, 6,7-dichloro-2-trifluoromethyl-2H-1-benzopyran-3-carboxylic acid, 6,8-dichloro-2-trifluoromethyl-2H-1-benzopyran-3-carboxylic acid, 2-trifluoromethyl-3H-naptho[2,1-b]pyran-3-carboxylic acid, 6-chloro-8-methyl-2-trifluoromethyl-2H-1-benzopyran-3-carboxylic acid, 8-chloro-6-methyl-2-trifluoromethyl-2H-1-benzopyran-3-carboxylic acid, 8-chloro-6-methoxy-2-trifluoromethyl-2H-1-benzopyran-3-carboxylic acid, 6-bromo-8-chloro-2-trifluoromethyl-2H-1-benzopyran-3-carboxylic acid, 8-bromo-6-fluoro-2-trifluoromethyl-2H-1-benzopyran-3-carboxylic acid, 8-bromo-6-methyl-2-trifluoromethyl-2H-1-benzopyran-3-carboxylic acid, 8-bromo-5-fluoro-2-trifluoromethyl-2H-1-benzopyran-3-carboxylic acid, 6-chloro-8-fluoro-2-trifluoromethyl-2H-1-benzopyran-3-carboxylic acid, 6-bromo-8-methoxy-2-trifluoromethyl-2H-1-benzopyran-3-carboxylic acid, 6-[[(phenylmethyl)amino]sulfonyl]-2-trifluoromethyl-2H-1-benzopyran-3-carboxylic acid, 6-[(dimethylamino)sulfonyl]-2-trifluoromethyl-2H-1-benzopyran-3-carboxylic acid, 6-[(methylamino)sulfonyl]-2-trifluoromethyl-2H-1-benzopyran-3-carboxylic acid, 6-[(4-morpholino)sulfonyl]-2-trifluoromethyl-2H-1-benzopyran-3-carboxylic acid, 6-[(1,1-dimethylethyl)aminosulfonyl]-2-trifluoromethyl-2H-1-benzopyran-3-carboxylic acid, 6-[(2-methylpropyl)aminosulfonyl]-2-trifluoromethyl-2H-1-benzopyran-3-carboxylic acid, 6-methylsulfonyl-2-trifluoromethyl-2H-1-benzopyran-3-carboxylic acid, 8-chloro-6-[[(phenylmethyl)amino]sulfonyl]-2-trifluoromethyl-2H-1-benzopyran-3-carboxylic acid, 6-phenylacetyl-2-trifluoromethyl-2H-1-benzopyran-3-carboxylic acid, 6,8-dibromo-2-trifluoromethyl-2H-1-benzopyran-3-carboxylic acid, 8-chloro-5,6-dimethyl-2-trifluoromethyl-2H-1-benzopyran-3-carboxylic acid, 6,8-dichloro-(S)-2-trifluoromethyl-2H-1-benzopyran-3-carboxylic acid, 6-benzylsulfonyl-2-trifluoromethyl-2H-1-benzopyran-3-carboxylic acid, 6-[[N-(2-furylmethyl)amino]sulfonyl]-2-trifluoromethyl-2H-1-benzopyran-3-carboxylic acid, 6-[[N-(2-phenylethyl)amino]sulfonyl]-2-trifluoromethyl-2H-1-benzopyran-3-carboxylic acid, 6-iodo-2-trifluoromethyl-2H-1-benzopyran-3-carboxylic acid, 7-(1,1-dimethylethyl)-2-pentafluoroethyl-2H-1-benzopyran-3-carboxylic acid, 6-chloro-2-trifluoromethyl-2H-1-benzothiopyran-3-carboxylic acid. 6-chloro-2-trifluoromethyl-2H-1-benzopyran-3-carboxylic acid, (S)-6-chloro-2-trifluoromethyl-2H-1-benzopyran-3-carboxylic acid, 6-chloro-7-(1,1-dimethylethyl)-2-trifluoromethyl-2H-1-benzopyran-3-carboxylic acid, (S)-6-chloro-7-(1,1-dimethylethyl)-2-(trifluoromethyl)-2H-1-benzopyran-3-carboxylic acid, 6-trifluoromethoxy-2-trifluoromethyl-2H-1-benzopyran-3-carboxylic acid, (S)-6-trifluoromethoxy-2-trifluoromethyl-2H-1-benzopyran-3-carboxylic acid, 6-formyl-2-(trifluoromethyl)-2H-1-benzopyran-3-carboxylic acid, 6-(difluoromethyl)-2-(trifluoromethyl)-2H-1-benzopyran-3-carboxylic acid, 6,8-dichloro-7-methyl-2-(trifluoromethyl)-2H-1-benzopyran-3-carboxylic acid, 6,8-dichloro-2-trifluoromethyl-2H-1-benzopyran-3-carboxylic acid, (S)-6,8-dichloro-2-(trifluoromethyl)-2H-1-benzopyran-3-carboxylic acid, 6-chloro-1,2-dihydro-2-(trifluoromethyl)-3-quinolinecarboxylic acid, (S)-6-chloro-1,2-dihydro-2-(trifluoromethyl)-3-quinolinecarboxylic acid, 6,8-dichloro-1,2-dihydro-2-(trifluoromethyl)-3-quinolinecarboxylic acid, 7-(1,1-dimethylethyl)-2-trifluoromethyl-2H-1-benzopyran-3-carboxylic acid, 6,7-dichloro-2-trifluoromethyl-2H-1-benzopyran-3-carboxylic acid, 5,6-dichloro-2-(trifluoromethyl)-2H-1-benzopyran-3-carboxylic acid, 2,6-bis(trifluoromethyl)-2H-1-benzopyran-3-carboxylic acid, 5,6,7-trichloro-2-(trifluoromethyl)-2H-1-benzopyran-3-carboxylic acid, 6,7,8-trichloro-2-(trifluoromethyl)-2H-1-benzopyran-3-carboxylic acid, 6-iodo-1,2-dihydro-2-(trifluoromethyl)-3-quinolinecarboxylic acid, 6-bromo-1,2-dihydro-2-(trifluoromethyl)-3-quinolinecarboxylic acid, 6-chloro-7-methyl-2-(trifluoromethyl)-2H-1-benzothiopyran-3-carboxylic acid, 6,8-dichloro-2-trifluoromethyl-2H-1-benzothiopyran-3-carboxylic acid, and mixtures thereof.

14. The method according to claim 12, wherein the chromene Cox-2 selective inhibitor comprises at least one compound that is chosen from:
(S)-6-chloro-7-(1,1-dimethylethyl)-2-(trifluoromethyl)-2H-1-benzopyran-3-carboxylic acid,
(2S)-6,8-dimethyl-2-(trifluoromethyl)-2H-chromene-3-carboxylic acid,
(2S)-6-chloro-8-methyl-2-(trifluoromethyl)-2H-chromene-3-carboxylic acid,
(2S)-8-ethyl-6-(trifluoromethoxy)-2-(trifluoromethyl)-2H-chromene-3-carboxylic acid,
(S)-6,8-dichloro-2-(trifluoromethyl)-2H-1-benzopyran-3-carboxylic acid, and
(2S)-6-chloro-5,7-dimethyl-2-(trifluoromethyl)-2H-chromene-3-carboxylic acid, and mixtures thereof.

15. The method according to claim 2, wherein the bone disorder treatment agent comprises an antiresorptive compound.

16. The method according to claim 2, wherein the bone disorder treatment agent comprises at least one compound that is chosen from bisphosphonates, calcitonin, estrogens, selective estrogen receptor modulators, parathyroid hormone, vitamins, and mixtures thereof.

17. The method according to claim 2, wherein the bone disorder treatment agent comprises bisphosphonates.

18. The method according to claim 2, wherein the bone disorder treatment agent comprises selective estrogen receptor modulators.

19. The method according to claim 2, wherein the bone disorder treatment agent comprises at least one compound that is chosen from osteoclast-secreted proteins; growth hormone secretagogues; integrin inhibitors (blockers); interleukin-6 inhibitors; interleukin-12 inhibitors; matrix metalloproteinase inhibitors; phosphodiesterase VII inhibitors; glycosylated polyamines; pyridine compounds; cysteine protease inhibitors; prostaglandin agonists; thiol-containing compounds; amino-alcohol derivatives; dipeptidyl peptidase-IV inhibitors; corticotrophin releasing factor antagonists; isoflavones; oxytocin; oxytocin analogs; retinoid antagonists; steroids; cytokines; polynucleotides; fibrinogen-binding inhibitors; antiresorptive agents; bicyclic amino acids; progestins; xanthine oxidase inhibitors; alphaᵥbeta₃ antagonists; selective estrogen receptor modulators; cathespin K inhibitors; ATP proton pump inhibitors; androgen receptor modulators; and mixtures thereof.

20. The method according to claim 2, wherein the bone disorder treatment agent comprises at least one compound that is chosen from 4-aminobutanoic acid derivatives; thienyl substituted acylguanidines; genus anethum extract; halogenated triphenylethylene derivatives; condensed 4,5,6,7-tetrahydrobenzo[C]thiophene, arylalkanoylpyridazine; disulfides; beta-amino acid nitrile; androst-5-ene-3.beta.,17.beta.-diol; N-(substituted glycyl)-2 cyanopyrrolidines; protein tyrosine phosphatase inhibitor; insulin-like growth factor I (IGF-I); 11.beta.-aryl-substituted 14,17-ethanoestratriene; B-nor-6-thiaequilenin; nitric oxide donors; nitric oxide synthase substrate; novel tripeptide and tetrapeptide analogs; novel heterocycles; novel purines; triphenylmethane derivatives; dietary supplement with vitamin D, calcium and osteoblast stimulant; 1,3-dihydroxy-20, 20-dialkyl-vitamin D3 analogs; novel pyridopyrimidones; novel quinazolines; novel quinolines; novel pyridopyrimidines; novel pyrazolo- and pyrrolo-pyrimidines; skeletal anabolic drugs; 3-desoxy vitamin D3 analogs; heterocyclic aromatic compounds useful as growth hormone secretagogues; quinolinones; estrogenic compounds; conjugated estrogens; novel peptide-like FPP-analogues; 3-desoxy-vitamin D3 analog esters; 1,3-dihydroxy-20,20-cycloalkyl-vitamin D3 analogs; prostaglandin conjugates; vitamin D3 analogs with bis C-20 side chains; fluorinated vitamin D3 analogs; methanediphosphonate derivatives; (4-arylsulfonylamino)-tetrahydropyran-4-carboxilic acid hydroxamide derivatives; 3-hydroxy-4-pyrone, epoxysuccinic acid derivatives, triaryl-thylene derivatives; 4-aryloxy-5-hydroxy-2(5H)-furanones; 3,4-diarylchromas; droloxifene; tamoxifen; 4-hydroxy-tamoxifen; toremeifene; levormeloxifene; idoxifene; 6-(4-hydroxy-phenyl)-5-[4-(2-piperidin-1-yl-ethoxy)-benzyl]-naphthalen-2ol, {4-[2-(2-aza-bicyclo[2.2. 1]hept-2-yl)-ethoxy]-phenyl}-[6-hydroxy-2-(4-hydroxy-phenyl)-benzo[b]thiophen-3-yl]-methanone; 3-(4-(1,2-diphenyl-but-1-enyl)-phenyl)-acrylic acid; 2-(4-methoxy-phenyl)-3-[4-(2-piperidin-1-yl-ethoxy)-phenoxy]-benzo[b]thiophen-6-ol; cis-6-(4-fluoro-phenyl)-5-[4-(2-piperidin-1-yl-ethoxy)-phenyl]-5,6,7,8-tetrahydro-naphthalene-2-ol; (−)-cis-6-phenyl-5-[4-(2-pyrrolidin-1-yl-ethoxy)-phenyl]-5,6,7,8-tetrahydro-naphthalene-2-ol; cis-6-phenyl-5-[4-(2-pyrrolidin-1-yl-ethoxy)-phenyl]-5,6,7,8-tetrahydro-naphthalene-2-ol; cis-1-[6′-pyrrolodinoethoxy-3′-pyridyl]-2-phenyl-6-hydroxy-1,2,3,4-tetrahydronaphthalene; 1-(4′-pyrrolidinoethoxyphenyl)-2-(4″-fluorophenyl)-6-hydroxy-1,2,3,4-tetrahydroisoquinoline; cis-6-(4-hydroxyphenyl)-5-[4-(2-piperidin-1-yl-ethoxy)-phenyl]-5,6,7,8-tetrahydro-naphthalene-2-ol; 1-(4′-pyrrolidinolethoxyphenyl)-2-phenyl-6-hydroxy-1,2,3,4-tetrahydroisoquinoline; β-alanine derivatives; centchroman; thienyl substituted acylguanidines; myb induced myeloid protein-1; fused thiophene derivates; benzene butyric acid; isoxazole; dibenzoazulene; 3-cyanoquinolines; 3-cyano-1,6-napthyridines; 3 cyano-1,7,-napthyridines; benzazepinone derivatives; fluorides; norethindrone; medroxyprogesterone; and mixtures thereof.

21. The method according to claim 16, wherein the bisphosphonate comprises at least one compound that is chosen from alendronate, mildronate, olpadronate, ibandronate, risedronate, etidronate, zoledronate, minodronate, tiludronate, cimadronate, incadronate, neridronate, pamidronate, clodronate, menodronate, and mixtures thereof.

22. The method according to claim 16, wherein the bisphosphonate comprises at least one compound that is chosen from:
4-amino-1-hydroxybutylidene-1,1-bisphosphonic acid,
N,N-dimethyl-3-amino-1-hydroxypropylidene-1,1-bisphosphonic acid,
1-hydroxy-3-(N-methyl-N-pentylamino)propylidene-1,1-bisphosphonic acid,
1-hydroxy-2-(3-pyridyl)ethylidene-1,1-bisphosphonic acid,
1-hydroxyethylidene-1,1-bisphosphonic acid,
1-hydroxy-3-(1-pyrrolidinyl)propylidene-1,1-bisphosphonic acid,
1-hydroxy-2-(1-imidazolyl)etylidene-1,1-bisphosphonic acid,
1-hydroxy-2-(imidazo[1,2-a]pyridin-3-yl)ethylidene-1,1-bisphosphonic acid,
1-(4-chlorophenylthio)methylidene-1,1-bisphosphonic acid,
1-(cycloheptylamino)methylidene-1,1-bisphosphonic acid,
6-amino-1-hydroxyhexylidene-1,1-bisphosphonic acid,
methanebisphosphonic acid, and mixtures thereof.

23. The method according to claim 16, wherein the estrogen comprises at least one compound that is chosen from estradiol, esterified estrogen, estropipate, ethinyl estradiol, conjugated estrogen, conjugated estrogen plus medroxyprogesterone acetate, and mixtures thereof.

24. The method according to claim 16, wherein the selective estrogen receptor modulator comprises raloxifene hydrochloride.

25. The method according to claim 16, wherein the vitamin comprises at least one compound that is chosen from calcium, vitamin D, and mixtures thereof.

26. The method according to claim 2, wherein the subject suffers from or is predisposed to bone disorders or bone disorder-related complications selected from the group consisting of osteopenia, osteoporosis, Paget's disease (osteitis deformans), bone degredation, bone weakening, skeletal distortion, low bone mineral density, scoliosis, osteomalacia, osteomyelitis, osteogenesis imperfecta, osteopetrosis, periodontal disease, enchondromatosis, osteochondromatosis, achondroplasia, avascular necrosis, fibrous dysplasia, hyperparathyroidism (osteitis fibrosa cystica), hypophosphatasia, fibrodysplasia ossificans progressive, bone fractures, bone breaks, and pain and/or inflammation of the bone.

27. The method according to claim 2, further comprising administering to the subject an amount of a Cox-2 inhibitor and an amount of a bone disorder treatment agent wherein the amount of the Cox-2 inhibitor and the amount of the bone disorder treatment agent together comprise a therapeutically effective amount.

28. A therapeutic composition comprising at least one Cox-2 inhibitor and one or more bone disorder treatment agents.

29. A pharmaceutical composition comprising a Cox-2 inhibitor, a bone disorder treatment agent, and a pharmaceutically acceptable carrier.

30. A kit comprising one dosage form comprising a Cox-2 inhibitor and a second dosage form comprising a bone disorder treatment agent.
